(19) United States
(12) Patent Application Publication
(10) Pub. No.: US 2007/0270428 A1
(43) Pub. Date: Nov. 22, 2007

(54) Use of Cyclooxygenase-2 Inhibitors for the Treatment of Depressive Disorders

(76) Inventors: James Hagan, Essex (GB); Emiliangelo Ratti, Verona (IT); Carol Routledge, Essex (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/595,800

(22) PCT Filed: Nov. 17, 2004
(86) PCT No.: PCT/EP04/13070

§ 371 Date: Feb. 21, 2007

(30) Foreign Application Priority Data
Nov. 19, 2003 (GB) 0326967.7
Dec. 2, 2003 (GB) 0327937.9
Jan. 28, 2004 (GB) 0401862.8

(51) Int. Cl. A61K 31/513; A61K 31/444; A61K 31/4525; A61P 25/24; A61K 31/5025

(52) U.S. Cl. 514/248; 514/274; 514/321; 514/334

(57) ABSTRACT

The invention concerns the use of compounds of formula (I), (II) and (III): 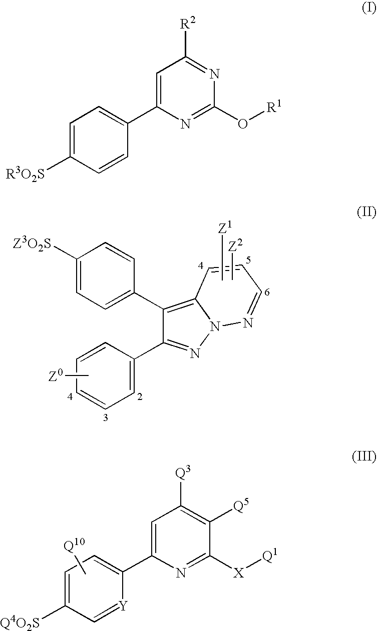 which are COX-2 (cyclooxygenase-2) inhibitors, and pharmaceutically acceptable salts or solvates thereof, for the treatment of depressive disorders in combination with an effective amount of a second component which is a selective serotonin reuptake inhibitor.

Depression is a chronic disease that affects persons of all ages. In the Diagnostic and Statistical Manual of Mental disorders, Fourth Edition, (DSM IV published by the American Psychiatric Association, depressive disorders are classified under mood disorders and are divided into three types: major depressive disorder, dysthymic disorder and depressive disorder not otherwise specified. Major depressive disorder and dysthymic disorder are differentiated based on chronicity, severity and persistence. In major depression, the depressed mood must be present for two weeks. In dysthymic disorder, the depressed mood must be present for two weeks. In dysthymic disorder the depressed mood must be present most days over a period of two years. Usually, major depressive disorder is characterized by its sharp contrast to usual functioning. A person with a major depressive episode can be functioning and feeling normal and suddenly develop severe symptoms of depression. By contrast, a person with dysthymic disorder has chronic depression with less severe symptoms than major depression.

In the context of the present invention the term depressive disorders encompasses, but it is not limited to, bipolar depression, bipolar depression I, bipolar depression II, unipolar depression, single or recurrent major depressive episodes with or without psychotic features, catatonic features, melancholic features, atypical features or postpartum onset, anxiety and panic disorders.

Other mood disorders encompassed within the term major depressive disorders include dysthymic disorder with early or late onset and with or without atypical features, neurotic depression, post traumatic stress disorders, post operative stress and social phobia; mood disorders induced by alcohol, amphetamines, cocaine, hallucinogens, inhalants, opioids, phencyclidine, sedatives, hypnotics, anxiolytics and other substances; and adjustment disorder with depressed mood. Major depressive disorders may also result from a general medical condition including, but not limited to, myocardial infarction, diabetes, miscarriage or abortion, etc.

In an effort to treat depression, a variety of antidepressant compositions have been developed. Among these the selective serotonin reuptake inhibitors (hereinafter referred to as SSRIs) have become first choice therapeutics in the treatment of depression, certain forms of anxiety and social phobias, because they are effective, well tolerated and have a favourable safety profile compared to the classic tricyclic antidepressants.

However, clinical studies on depression indicate that non-response to SSRIs is substantial, up to 30%. Another, often neglected, factor in antidepressant treatment is compliance, which has a rather profound effect on the patient's motivation to continue pharmacotherapy.

First of all, there is the delay in therapeutic effect of SSRIs. Sometimes symptoms even worsen during the first weeks of treatment. Without addressing these problems, real progress in the pharmacotherapy of depression and anxiety disorders is not likely to happen.

Accordingly, the development of an antidepressant capable of exhibiting its effect rapidly is desired.

The invention provides a method for treating a patient suffering from or susceptible to psychiatric disorders as defined above comprising administering to said patient an effective amount of a first component which is a COX-2 inhibitor, in combination with an effective amount of a second component which is a serotonin reuptake inhibitor.

In the general expressions of the present invention, the first component is a compound which acts as a COX-2 (cyclooxygenase 2) inhibitor.

In one embodiment, the present invention provides a new use of compounds of formula (I) 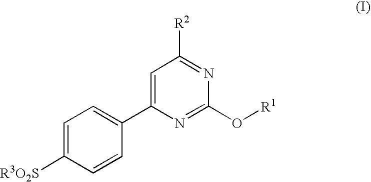
and pharmaceutically acceptable salts or solvates thereof, wherein
R¹ is selected from the group consisting of H, C₁₋₆alkyl, C₁₋₂alkyl substituted by one to five fluorine atoms, C₃₋₆alkenyl, C₃₋₆alkynyl, C₃₋₁₀cycloalkylC₀₋₆alkyl, C₄₋₁₂bridged cycloalkyl, A(CR⁴R⁵)ₙ and B(CR⁴R⁵)ₙ;
R² is C₁₋₂alkyl substituted by one to five fluorine atoms;
R³ is selected from the group consisting of C₁₋₆alkyl, NH₂ and R⁷CONH;
R⁴ and R⁵ are independently selected from H or C₁₋₆alkyl;
A is selected from the group consisting of unsubstituted 5- or 6-membered heteroaryl, unsubstituted 6-membered aryl, 5- or 6-membered heteroaryl substituted by one or more R⁶ and 6-membered aryl substituted by one or more R⁶;
R⁶ is selected from the group consisting of halogen, C₁₋₆alkyl, C₁₋₆alkyl substituted by one more fluorine atoms, C₁₋₆alkoxy, C₁₋₆alkoxy substituted by one or more F, NH₂SO₂ and C₁₋₆alkylSO₂;
B is a ring selected from the group consisting of 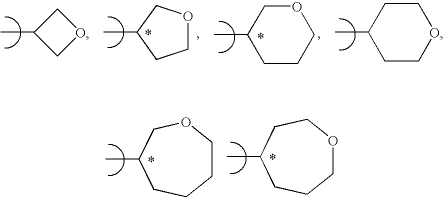
   where z,1 defines the point of attachment of the ring;
R⁷ is selected from the group consisting of H, C₁₋₆alkyl, C₁₋₆alkoxy, C₁₋₆alkylOC₁₋₆alkyl, phenyl, HO₂CC₁₋₆alkyl, C₁₋₆alkylOCOC₁₋₆alkyl, C₁₋₆alkylOCO, H₂NC₁₋₆alkyl, C₁₋₆alkylOCONHC₁₋₆alkyl and C₁₋₆alkylCONHC₁₋₆alkyl; and
n is 0 to 4.

The term halogen is used to represent fluorine, chlorine, bromine or iodine.

The term “alkyl” as a group or part of a group means a straight or branched chain alkyl group, for example a methyl, ethyl, n-propyl, i-propyl, n-butyl, s-butyl or t-butyl group.

The term 5-membered heteroaryl means a heteroaryl selected from the following:

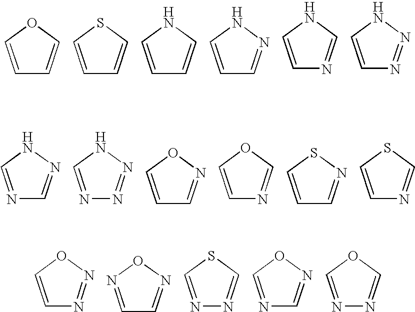

The term 6- membered heteroaryl means a heteroaryl selected from the following:

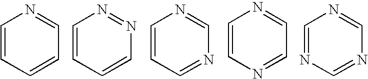

The term 6-membered aryl means:

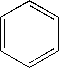

It is to be understood that the present invention encompasses all isomers of the compounds of formula (I) and their pharmaceutically acceptable derivatives, including all geometric, tautomeric and optical forms, and mixtures thereof (e.g. racemic mixtures). In particular when the ring B lacks a plane of symmetry the compounds of formula (I) contain a chiral centre as indicated therein by the asterisk *. Furthermore, it will be appreciated by those skilled in the art that when R⁴ and R⁵ in formula (I) are different the corresponding compounds contain at least one chiral centre, by virtue of the asymmetric carbon atom defined thereby, and that such compounds exist in the form of a pair of optical isomers (i.e. enantiomers).

In one aspect of the invention R¹ is selected from the group consisting of H, C₁₋₆alkyl, C₁₋₂alkyl substituted by one to five fluorine atoms, C₃₋₆alkenyl, C₃₋₆alkynyl, C₃₋₁₀cycloalkylC₀₋₆alkyl, C₄₋₁₂bridged cycloalkyl and B(CR⁴R⁵)ₙ;

In another aspect of the invention R¹ is C₁₋₆alkyl or C₁₋₂alkyl substituted by one to five fluorine atoms. In another aspect R¹ is C₂₋₆alkyl (e.g. n-butyl).

In another aspect of the invention R¹ is C₃₋₁₀cycloalkylC₀₋₆alkyl, such as C₃₋₁₀cycloalkyl (e.g. cyclopentyl or cyclohexyl). In another aspect R¹ is C₃₋₁₀cycloalkylmethyl, such as C₃₋₇cycloalkylmethyl (e.g. cyclopentylmethyl).

In another aspect of the invention R¹ is A(CR⁴R⁵)ₙ.

In another aspect of the invention R² is CHF₂, CH₂F or CF₃. In another aspect R² is CF₃.

In another aspect of the invention R³ is C₁₋₆alkyl, such as C₁₋₃alkyl (e.g. methyl).

In another aspect of the invention R⁴ and R⁵ are independently selected from H or methyl. In another aspect R⁴ and R⁵ are both H.

In another aspect of the invention A is selected from the group consisting of 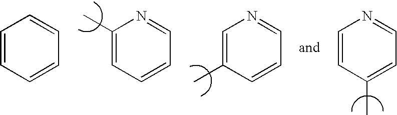
where [symbol] defines the point of attachment of the ring and A is unsubstituted or substituted by one or two R⁶.

In another aspect of the invention R⁶ is selected from the group consisting of halogen (e.g. F), C₁₋₃alkyl (e.g. methyl), C₁₋₃alkyl substituted by one to three fluorine atoms (e.g. CF₃), and C₁₋₃alkoxy (e.g. methoxy).

In another aspect of the invention R⁷ is selected from the group consisting of C₁₋₆alkyl (e.g. ethyl), phenyl and aminomethyl.

In another aspect of the invention n is 1 to 4.

In another aspect of the invention n is 0 to 2 (e.g. 0).

It is to be understood that the invention covers all combinations of particular aspects of the invention as described hereinabove.

Within the invention there is provided one group of compounds of formula (I) (group A) wherein: R¹ is C₁₋₆alkyl (e.g. n-butyl); R² is CF₃; and R³ is C₁₋₆alkyl, such as C₁₋₃alkyl (e.g. methyl).

Within the invention there is provided another group of compounds of formula (I) (group B) wherein: R¹ is C₃₋₁₀cycloalkylC₀₋₆alkyl, such as C₃₋₁₀cycloalkyl (e.g. cyclopentyl or cyclohexyl); R² is CF₃; and R³ is C₁₋₆alkyl, such as C₁₋₃alkyl (e.g. methyl).

Within the invention there is provided another group of compounds of formula (I) (group C) wherein: R¹ is C₃₋₁₀cycloalkylmethyl, such as C₃₋₇cycloalkylmethyl (e.g. cyclopentylmethyl); R² is CF₃; and R³ is C₁₋₆alkyl, such as C₁₋₃alkyl (e.g. methyl).

Within the invention there is provided another group of compounds of formula (I) (group D) wherein: R¹ is A(CR⁴R⁵)ₙ; R² is CF₃; R³ is C₁₋₆alkyl, such as C₁₋₃alkyl (e.g. methyl); R⁴ and R⁵ are independently selected from H or methyl; A is selected from the group consisting of 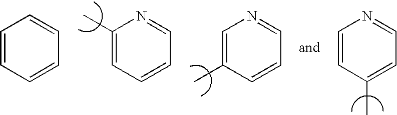
and A is unsubstituted or substituted by one or two R⁶; R⁶ is selected from the group consisting of halogen (e.g. F), C₁₋₃alkyl (e.g. methyl), C₁₋₃alkyl substituted by one to three fluorine atoms (e.g. CF₃), and C₁₋₃alkoxy (e.g. methoxy); and n is 0 to 2 (e.g. 0).

Within group D, there is provided a further group of compounds (group D1) wherein: R¹ is A(CR⁴R⁵)ₙ; R² is CF₃; R³ is methyl; R⁴ and R⁵ are both H; A is selected from the group consisting of 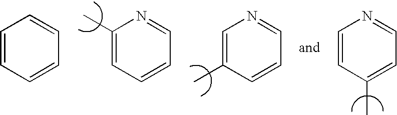
and A is unsubstituted or substituted by one or two R⁶; R⁶ is selected from the group consisting of fluorine, chlorine, methyl, CF₃ and methoxy; and n is 0 or 1.

Compounds of formula (I) and salts and solvates thereof are described in PCT publication No. WO02/096885, published 5 Dec. 2002 and U.S. application Ser. No. 10/477,547, published 2 Sep. 2004. The disclosures of these references are incorporated herein by reference in their entirety. Compounds of formula (I) may be prepared by any method described in WO 021096885, U.S. application Ser. No. 10/477,547 and equivalent patent applications.

In a further embodiment, the present invention provides compounds of formula (I) and pharmaceutically acceptable salts or solvates thereof for use in the preparation of a medicament for the treatment of depressive disorders as defined above.

In another embodiment, the present invention a method for the treatment of bipolar disorder, bipolar depression, bipolar disorder I, bipolar disorder II, unipolar depression comprising administering a therapeutically effective amount an effective amount of a first component which is a compound of formula (I) and pharmaceutically acceptable salts or solvates thereof, in combination with an effective amount of a second component which is a selective serotonin reuptake inhibitor.

In one embodiment, the present invention provides a new use of compounds of formula (II) 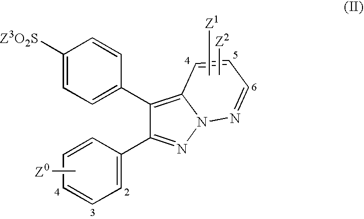
and pharmaceutically acceptable salts or solvates thereof, wherein
Z⁰ is selected from the group consisting of halogen, C₁₋₆alkyl, C₁₋₆alkoxy, C₁₋₆alkoxy substituted by one or more fluorine atoms, and O(CH₂)ₙNZ⁴Z⁵;
Z¹ and Z² are each the same or different and are independently selected from the group consisting of H, C₁₋₆alkyl, C₁₋₆alkyl substituted by one or more fluorine atoms, C₁₋₆alkoxy, C₁₋₆hydroxyalkyl, SC₁₋₆alkyl, C(O)H, C(O)C₁₋₆alkyl, C₁₋₆alkylsulphonyl, C₁₋₆alkoxy substituted by one or more fluorine atoms, O(CH₂)ₙCO₂C₁₋₆alkyl, O(CH₂)ₙSC₁₋₆alkyl, (CH₂)ₙNZ⁴Z⁵, (CH₂)ₙSC₁₋₆alkyl and C(O)NZ⁴Z⁵;
with the proviso that when Z⁰ is at the 4-position and is halogen, then at least one of Z¹ and Z² is C₁₋₆alkylsulphonyl, C₁₋₆alkoxy substituted by one or more fluorine atoms, O(CH₂)ₙCO₂C₁₋₆alkyl, O(CH₂)ₙSC₁₋₆alkyl, (CH₂)ₙNZ⁴Z⁵, (CH₂)ₙSC₁₋₆alkyl or C(O)NZ⁴Z⁵;
Z³ is C₁₋₆alkyl or NH₂;
Z⁴ and Z⁵ are each the same or different and are independently selected from the group consisting of H, or C₁₋₆alkyl or, Z⁴ and Z⁵ together with the nitrogen atom to which they are bound, form a 4-8 membered saturated heterocyclic ring having 1 or 2 heteroatoms selected from N, O and S; and
n¹ is 1-4.

The term halogen is used to represent fluorine, chlorine, bromine or iodine.

The term ‘alkyl’ as a group or part of a group means a straight or branched chain alkyl group, for example a methyl, ethyl, n-propyl, i-propyl, n-butyl, s-butyl or t-butyl group.

Preferably, Z⁰ is at the 3- or 4-position of the phenyl ring, as defined in formula (I).

Preferably, Z¹ is at the 6-position of the pyridazine ring, as defined in formula (I).

Preferably, Z⁰ is F, C₁₋₃alkyl, C₁₋₃alkoxy, C₁₋₃alkoxy substituted by one or more fluorine atoms, or O(CH₂)₁₋₃NZ⁴Z⁵. More preferably Z⁰ is F, C₁₋₃alkoxy or C₁₋₃alkoxy substituted by one or more fluorine atoms.

Preferably, Z¹ is C₁₋₄alkylsulphonyl, C₁₋₄alkoxy substituted by one or more fluorine atoms, O(CH₂)₁₋₃CO₂C₁₋₄alkyl, O(CH₂)₁₋₃SC₁₋₄alkyl, (CH₂)₁₋₃NZ⁴Z⁵, (CH₂)₁₋₃SC₁₋₄alkyl or C(O)NZ⁴Z⁵ or, when Z⁰ is C₁₋₆alkyl, C₁₋₆alkoxy, O(CH₂)ₙNZ⁴Z⁵, may also be H. More preferably Z¹ is C₁₋₄alkylsulphonyl, C₁₋₄alkoxy substituted by one or more fluorine atoms or, when Z⁰ is C₁₋₆alkyl, C₁₋₆alkoxy, C₁₋₄alkoxy substituted by one or more fluorine atoms, or O(CH₂)ₙNZ⁴Z⁵, may also be H.

Preferably, Z² is H.

Preferably, Z³ is methyl or NH₂.

Preferably Z⁴ and Z⁵ are independently C₁₋₃alkyl or, together with the nitrogen atom to which they are attached, form a 5-6 membered saturated ring.

Preferably, n is 1-3, more preferably 1 or 2.

Within the invention there is provided one group of compounds of formula (I) (group A1) and pharmaceutically acceptable salts or solvates thereof, wherein: Z⁰ is F, C₁₋₃alkyl, C₁₋₃alkoxy, C₁₋₃alkoxy substituted by one or more fluorine atoms, or O(CH₂)ₙNZ⁴Z⁵; Z¹ is C₁₋₄alkylsulphonyl, C₁₋₄alkoxy substituted by one or more fluorine atoms, O(CH₂)ₙCO₂C₁₋₃alkyl, O(CH₂)ₙSC₁₋₄alkyl, (CH₂)ₙNZ⁴Z⁵, (CH₂)ₙSC₁₋₄alkyl or C(O)NZ⁴Z⁵ or, when Z⁰ is C₁₋₃alkyl, C₁₋₃alkoxy, C₁₋₃alkoxy substituted by one or more fluorine atoms, or O(CH₂)ₙNZ⁴Z⁵, may also be H; Z² is H; R³ is methyl or NH₂; Z⁴ and Z⁵ are independently C₁₋₃alkyl or, together with the nitrogen atom to which they are attached, form a 5-6 membered saturated ring; and n is 1-3.

Within group A, there is provided another group of compounds (group A2) and pharmaceutically acceptable salts or solvates thereof, wherein Z⁰ is F, methyl, C₁₋₂alkoxy, OCHF₂, or O(CH₂)ₙNZ⁴Z⁵; Z¹ is methylsulphonyl, OCHF₂, O(CH₂)ₙCO₂C₁₋₄alkyl, O(CH₂)ₙSCH₃, (CH₂)ₙNZ⁴Z⁵, (CH₂)ₙSCH₃ or C(O)NZ⁴Z⁵ or, when Z⁰ is methyl, C₁₋₂alkoxy, OCHF₂, or O(CH₂)ₙN(CH₃)₂, may also be H; Z² is H; Z³ is methyl or NH₂; Z⁴ and Z⁵ are both methyl or, together with the nitrogen atom to which they are attached, form a 5-6 membered saturated ring; and n is 1-2.

Within group A, there is provided a further group of compounds (group A3) wherein Z⁰ is F, C₁₋₃alkoxy or C₁₋₃alkoxy substituted by one or more fluorine atoms; Z¹ is C₁₋₄alkylsulphonyl, C₁₋₄alkoxy substituted by one or more fluorine atoms or, when Z⁰ C₁₋₃alkoxy or C₁₋₃alkoxy substituted by one or more fluorine atoms, may also be H; Z² is H; and Z³ is methyl or NH₂.

Within groups A, A2 and A3, Z⁰ is preferably at the 3- or 4-position of the phenyl ring and Z¹ is preferably at the 6-position of the pyridazine ring.

Compounds of formula (II) and salts and solvates thereof are described in PCT publication No. WO 99/12930, published 18 Mar. 1999 and U.S. Pat. No. 6,451,794, US-A-2003-0040517 and US-A-2003-0008872. The disclosures of these references are incorporated herein by reference in their entirety. Compounds of formula (II) may be prepared by any method described in WO 99/12930, U.S. Pat. No. 6,451,794, US-A-2003-0040517 and US-A-2003-0008872 and equivalent patent applications.

In a further embodiment, the present invention provides compounds of formula (II) and pharmaceutically acceptable salts or solvates thereof for use in the preparation of a medicament for the treatment of depressive disorders as defined above.

In another embodiment, the present invention a method for the treatment of bipolar disorder, bipolar depression, bipolar disorder I, bipolar disorder II, unipolar depression comprising administering a therapeutically effective amount an effective amount of a first component which is a compound of formula (II) and pharmaceutically acceptable salts or solvates thereof, in combination with an effective amount of a second component which is a selective serotonin reuptake inhibitor.

In one embodiment the present invention provides a new use of compounds of formula (III) 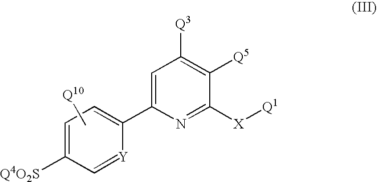
and pharmaceutically acceptable salts or solvates thereof, wherein:
X is selected from the group consisting of oxygen or NQ²;
Y is selected from the group consisting of CH or nitrogen;
Q¹ is selected from the group consisting of H, C₁₋₆alkyl, C₁₋₂alkyl substituted by one to five fluorine atoms, C₁₋₃alkylOC₁₋₃alkyl, C₃₋₆alkenyl, C₃₋₆alkynyl, C₃₋₁₀cycloalkylC₀₋₆alkyl, C₄₋₇cycloalkyl substituted by C₁₋₃alkyl or C₁₋₃alkoxy, C₄₋₁₂bridged cycloalkyl, A(CR⁶R⁷)ₙ and B(CR⁶R⁷)ₙ;
Q² is selected from the group consisting of H and C₁₋₆alkyl; or
Q¹ and Q² together with the nitrogen atom to which they are attached form a 4-8 membered saturated heterocyclic ring such as a pyrrolidine, morpholine or piperidine ring, or a 5-membered heteroaryl ring which is unsubstituted or substituted by one R⁸;
Q³ is selected from the group consisting of C₁₋₅alkyl and C₁₋₂alkyl substituted by one to five fluorine atoms;
Q⁴ is selected from the group consisting of C₁₋₆alkyl, NH₂ and R⁹CONH;
Q⁵ is selected from the group consisting of hydrogen, C₁₋₃alkyl, C₁₋₂alkyl substituted by one to five fluorine atoms, C₁₋₃alkylO₂C, halogen, cyano, (C₁₋₃alkyl)₂NCO, C₁₋₃alkylS and C₁₋₃alkylO₂S;
Q⁶ and Q⁷ are independently selected from H or C₁₋₆alkyl;
A¹ is an unsubstituted 5- or 6-membered heteroaryl or an unsubstituted 6-membered aryl, or a 5- or 6-membered heteroaryl or a 6-membered aryl substituted by one or more R⁸;
Q⁸ is selected from the group consisting of halogen, C₁₋₆alkyl, C₁₋₆alkyl substituted by one more fluorine atoms, C₁₋₆alkoxy, C₁₋₆alkoxy substituted by one or more F, NH₂SO₂ and C₁₋₆alkylSO₂;
B¹ is selected from the group consisting of 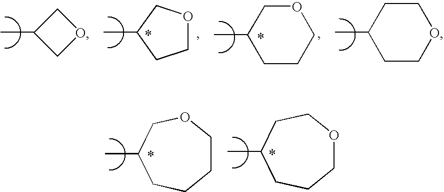
and where [symbol] defines the point of attachment of the ring;
Q⁹ is selected from the group consisting of H, C₁₋₆alkyl, C₁₋₆alkoxy, C₁₋₆alkylOC₁₋₆alkyl, phenyl, HO₂CC₁₋₆alkyl, C₁₋₆alkylOCOC₁₋₆alkyl, C₁₋₆alkylOCO, H₂NC₁₋₆alkyl, C₁₋₆alkylOCONHC₁₋₆alkyl and C₁₋₆alkyl CONHC₁₋₆alkyl;
Q¹⁰ is selected from the group consisting of H and halogen; and
n is 0 to 4;

The term ‘halogen’ is used to represent fluorine, chlorine, bromine or iodine.

The term ‘alkyl’ as a group or part of a group means a straight or branched chain alkyl group, for example a methyl, ethyl, n-propyl, i-propyl, n-butyl, s-butyl or t-butyl group.

The term ‘saturated heterocyclic’ means a saturated ring containing at least one atom other than carbon.

The term ‘5-membered heteroaryl’ means a heteroaryl selected from the following:

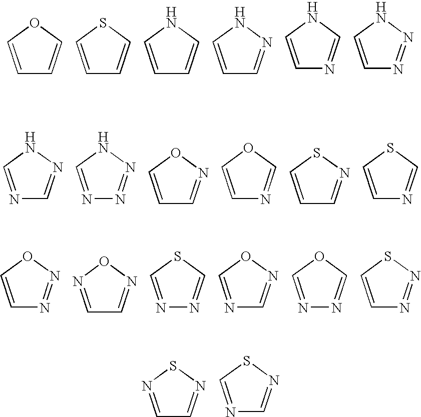

The term ‘6- membered heteroaryl’ means a heteroaryl selected from the following:

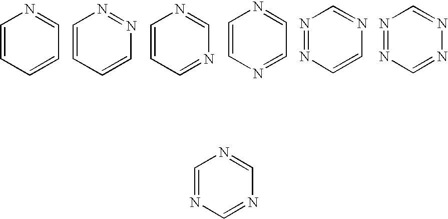

The term ‘6-membered aryl’ means:

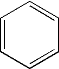

Compound of formula (III) may be a compound of formula (IIIC) 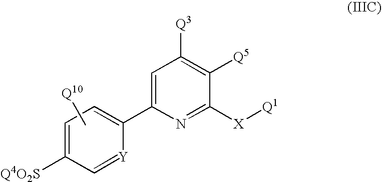
and pharmaceutically acceptable salts or solvates thereof, wherein
X is selected from the group consisting of oxygen or NR²;
Y is selected from the group consisting of CH or nitrogen;
Q¹ is selected from the group consisting of H, C₁₋₆alkyl, C₁₋₂alkyl substituted by one to five fluorine atoms, C₁₋₃alkylOC₁₋₃alkyl, C₃₋₆alkenyl, C₃₋₆alkynyl, C₃₋₁₀cycloalkylC₀₋₆alkyl, C₄₋₁₂bridged cycloalkyl, A(CQ⁶Q⁷)ₙ and B(CQ⁶Q⁷)ₙ;
Q² is selected from the group consisting of H and C₁₋₆alkyl; or
Q¹ and Q² together with the nitrogen atom to which they are attached form a 4-8 membered saturated heterocyclic ring such as a pyrrolidine, morpholine or piperidine ring;
Q³ is selected from the group consisting of C₁₋₅alkyl and C₁₋₂alkyl substituted by one to five fluorine atoms;
Q⁴ is selected from the group consisting of C₁₋₆alkyl, NH₂ and Q⁹CONH;
Q⁵ is selected from the group consisting of hydrogen, C₁₋₃alkyl, C₁₋₂alkyl substituted by one to five fluorine atoms, halogen, cyano, (C₁₋₃alkyl)₂NCO, C₁₋₃alkylS and C₁₋₃alkylO₂S;
Q⁶ and Q⁷ are independently selected from H or C₁₋₆alkyl;
A¹ is an unsubstituted 5- or 6-membered heteroaryl or an unsubstituted 6-membered aryl, or a 5- or 6-membered heteroaryl or a 6-membered aryl substituted by one or more Q⁸;
Q⁸ is selected from the group consisting of halogen, C₁₋₆alkyl, C₁₋₆alkyl substituted by one more fluorine atoms, C₁₋₆alkoxy, C₁₋₆alkoxy substituted by one or more F, NH₂SO₂ and C₁₋₆alkylSO₂;
B¹ is selected from the group consisting of 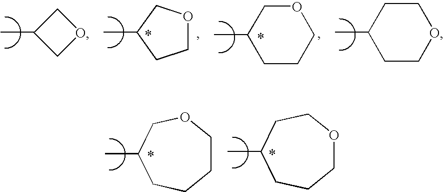
and where [symbol] defines the point of attachment of the ring;
Q⁹ is selected from the group consisting of H, C₁₋₆alkyl, C₁₋₆alkoxy, C₁₋₆alkylOC₁₋₆alkyl, phenyl, HO₂C₁₋₆alkyl, C₁₋₆alkylOCOC₁₋₆alkyl, C₁₋₆alkylOCO, H₂NC₁₋₆alkyl, C₁₋₆alkylOCONHC₁₋₆alkyl and C₁₋₆alkyl CONHC₁₋₆alkyl;
Q¹⁰ is selected from the group consisting of H and halogen; and
n is 0 to 4.

Compound of formula (III) may be a compound of formula (IIID) 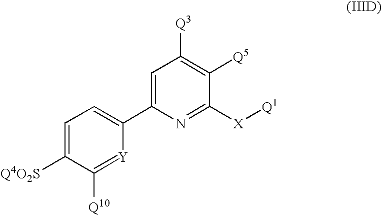
and pharmaceutically acceptable salts or solvates thereof, wherein all substituents are as for a compound of formula (III) defined hereinabove.

Compound of formula (III) may be a compound of formula (IIIE) 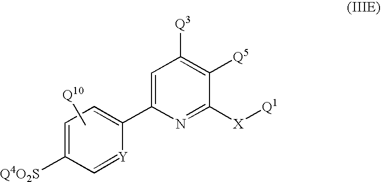
and pharmaceutically acceptable salts or solvates thereof, wherein
X is selected from the group consisting of oxygen or NQ²;
Y is selected from the group consisting of CH or nitrogen;
Q¹ is selected from the group consisting of H, C₁₋₆alkyl, C₁₋₂alkyl substituted by one to five fluorine atoms, C₁₋₃alkylOC₁₋₃alkyl, C₃₋₆alkenyl, C₃₋₆alkynyl, C₃₋₁₀cycloalkylC₀₋₆alkyl, C₄₋₇cycloalkyl substituted by C₁₋₃alkyl or C₁₋₃alkoxy, C₄₋₁₂bridged cycloalkyl, A(CR⁶R⁷)ₙ and B(CR⁶R⁷)ₙ;
Q² is selected from the group consisting of H and C₁₋₆alkyl; or
Q¹ and Q² together with the nitrogen atom to which they are bound form a 4-8 membered saturated heterocyclic ring or a 5-membered heteroaryl ring heteroaryl ring is unsubstituted or substituted by one R⁸; Q³ is selected from the group consisting of C₁₋₅alkyl and C₁₋₂alkyl substituted by one to five fluorine atoms;
Q⁴ is selected from the group consisting of C₁₋₆alkyl, NH₂ and R⁹CONH;
Q⁵ is selected from the group consisting of hydrogen, C₁₋₃alkyl, C₁₋₂alkyl substituted by one to five fluorine atoms, C₁₋₃alkylO₂C, halogen, cyano, (C₁₋₃alkyl)₂NCO, C₁₋₃alkylS and C₁₋₃alkylO₂S;
Q⁶ and Q⁷ are independently H or C₁₋₆alkyl;
A¹ is selected from the group consisting of unsubstituted 5- or 6-membered heteroaryl unsubstituted 6-membered aryl, 5- or 6-membered heteroaryl substituted by one or more R⁸; and 6-membered aryl substituted by one or more R⁸;
Q⁸ is selected from the group consisting of halogen, C₁₋₆alkyl, C₁₋₆alkyl substituted by one more fluorine atoms, C₁₋₆alkoxy, C₁₋₆alkoxy substituted by one or more F, NH₂SO₂ and C₁₋₆alkylSO₂;
B¹ is a ring selected from the group consisting of 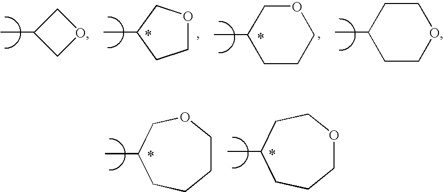
and where [symbol] defines the point of attachment of the ring;
Q⁹ is selected from the group consisting of H, C₁₋₆alkyl, C₁₋₆alkoxy, C₁₋₆alkylOC₁₋₆alkyl, phenyl, HO₂CC₁₋₆alkyl, C₁₋₆alkylOCOC₁₋₆alkyl, C₁₋₆alkylOCO, H₂NC₁₋₄alkyl, C₁₋₆alkylOCONHC₁₋₆alkyl and C₁₋₆alkylCONHC₁₋₆alkyl;
Q¹⁰ is selected from the group consisting of H and halogen; and
n is 0 to 4.

In another aspect of the invention Y is carbon.

In another aspect of the invention Q¹ is selected from the group consisting of, C₁₋₄alkyl, C₃₋₁₀cycloalkylC₀₋₆alkyl, C₅₋₆cycloalkyl substituted by C₁₋₂alkyl or C₁₋₂alkoxy, C₁₋₃alkylOC₁₋₃alkyl and C₁₋₂alkyl substituted by one to five fluorine atoms.

Representative examples of Q¹ include cyclohexylmethyl, cyclohexyl, n-butyl, n-pentyl, cyclopentyl, 2-methylpropyl, 2,2-dimethylpropyl, 2,2,2-trifluoroethyl, 2-methoxyethyl and ethyl.

Further representative examples of Q¹ include 1-methylethyl, 1-ethylpropyl, cycloheptyl, cis-4-methylcyclohexyl, trans-4-methylcyclohexyl, cyclobutyl, cyclopentanemethyl, and trans-4-(ethoxy)cyclohexyl.

In another aspect of the invention Q¹ is selected from the group consisting of A¹(CQ⁶Q⁷)ₙ and B¹(CQ⁶Q⁷)ₙ.

Further representative examples of Q¹ include benzyl, 4-chlorobenzyl, 2-furylmethyl, 4-methylphenyl, 4-fluorophenyl, 4-methoxyphenyl, 3-pyridyl, 2-chlorophenyl, 3,5-difluorobenzyl, 3-pyridylmethyl, 2-methylbenzyl, 2-chlorobenzyl, (S)-α-methylbenzyl, (R)-α-methylbenzyl, 6-methylpyridin-3-yl, 4-methoxybenzyl, 4-fluorobenzyl, 2-(5-methylfuryl)methyl, 4-methylbenzyl, 4-pyridylmethyl, 2-pyridylmethyl, 2-(6-methylpyridine)methyl, 2-thiophenylmethyl, 4-pyranylmethyl, 2-tetrahydrofurylmethyl, 2-(5-methylpyrazine)methyl and 4-ethoxybenzyl. Further representative examples of Q¹ include 1H-imidazol-2-ylmethyl, 1H-pyrazol-4-ylmethyl, (1-methyl-1H-imidazol-2-yl)methyl, (3-methyl-1H-pyrazol-4-yl)methyl, (1-methyl-1H-pyrazol-3-yl)methyl, (1-methyl-1H-pyrazol-4-yl)methyl, (3-methyl-1H-pyrazol-5-yl)methyl, (1-methyl-1H-pyrazol-5-yl)methyl, (1-methyl-1H-1,2,4-triazol-5-yl)methyl, (5-methyl-3-isoxazolyl)methyl, tetrahydro-2H-pyran-4-yl, tetrahydro-2H-pyran-4-ylmethyl, (6-methyl-3-pyridyl)methyl, 2-pyrazinylmethyl, (2-methyl-1H-imidazol4-yl)methyl, (4-methyl-1H-imidazol-5-yl)methyl, (4-methyl-1H-imidazol-2-yl)methyl, (1-ethyl-1H-imidazol-2-yl)methyl, (1,3-dimethyl-1H-pyrazol-4-yl)methyl, (1,5-dimethyl-1H-pyrazol4-yl)methyl, (3-methyl-5-isothiazolyl)methyl, (4-methyl-1,3-thiazol-2-yl)methyl, (3-methyl-4-isothiazolyl)methyl, [1-(fluoromethyl)-1H-pyrazol-4-yl]methyl, (2-methyl-3-pyridyl)methyl, (6-methyl-3-pyridyl)methyl, (1-methyl-1H-imidazol-2-yl)methyl, (5-chloro-2-pyridyl)methyl, 1H-imidazol-2-ylmethyl, 4-ethoxyphenyl, 3-chloro-4-methylphenyl, (5-chloro-2-pyridyl)methyl, (6-methyl-3-pyridyl)methyl, 2-methyl-3-pyridyl, 6-methyl-2-pyridyl, 2-pyrazinylmethyl, 2,6-dimethyl-3-pyridyl, 3,4-dichlorobenzyl, 5-chloro-3-pyridyl, 6-chloro-3-pyridazinyl, 3,5-dichlorobenzyl, 2-carboxyphenyl, (5-methyl-2-pyridyl)methyl, 4-chloro-3-(trifluoromethyl)benzyl, (5-bromo-2-pyridyl)methyl, (4-bromo-4-pyridyl)methyl, (3-methyl-4-isoxazolyl)methyl, 5-pyrimidinylmethyl, (3-methyl-1,2,4-oxadiazol-5-yl)methyl, (5-methyl-1,2,4-oxadiazol-3-yl)methyl and (1-ethyl-1H-1,2,4-triazol-5-yl)methyl.

In another aspect of the invention Q¹ is selected from the group consisting of C₃₋₆alkenyl and C₃₋₆alkynyl.

Further representative examples of Q¹ include propargyl and allyl.

In another aspect of the invention Q² is H or C₁₋₂alkyl.

Representative examples of Q² include H, methyl and ethyl.

In another aspect of the invention Q³ is CHF₂, CH₂F, CF₃or C₁₋₄alkyl.

Representative examples of Q³ include CF₃, CH₃ and ethyl.

Further representative examples of Q³ include CH₂F.

In another aspect of the invention Q⁴ is C₁₋₆alkyl, such as C₁₋₃alkyl.

Representative examples of Q⁴ include CH₃.

In another aspect of the invention Q⁴ is NH₂.

Further representative examples of Q⁴ include NH₂.

In another aspect of the invention Q⁵ is hydrogen or C₁₋₃alkyl.

Representative examples of Q⁵ include H or CH₃.

In another aspect of the invention R⁵ is CN, halogen or CO₂Et.

Further representative examples of Q⁵ include CN, F, Cl, CO₂Et.

In another aspect of the invention Q⁶ and Q⁷ are independently selected from H or methyl. In another aspect Q⁶ and Q⁷ are both H.

In another aspect of the invention A¹ is selected from the group consisting of 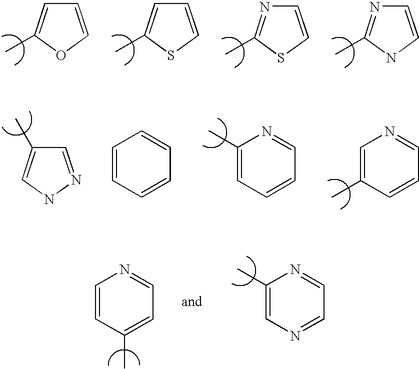
where [symbol] defines the point of attachment of the ring
and A¹ is unsubstituted or substituted by one or two Q⁸.

In another aspect of the invention A¹ is selected from the group consisting of

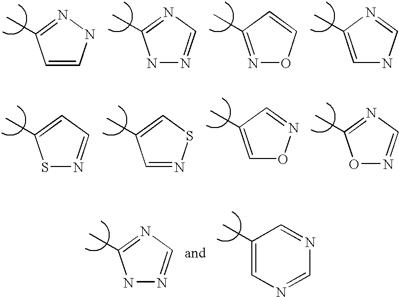

where [symbol] defines the point of attachment of the ring

In another aspect of the invention Q⁸ is selected from the group consisting of halogen, C₁₋₃alkyl, C₁₋₃alkyl substituted by one to three fluorine atoms (e.g. CF₃), and C₁₋₃alkoxy.

Representative examples of Q⁸ include F, Cl, CH₃, methoxy and ethoxy.

Further representative examples of Q⁸ include ethyl, fluoromethyl, CF₃ and Br.

Representative examples of B¹ include

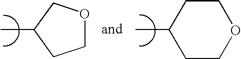

In another aspect of the invention Q⁹ is selected from the group consisting of C₁₋₆alkyl (e.g. ethyl), phenyl and aminomethyl.

In another aspect of the invention Q¹⁰ is H.

In another aspect of the invention in compounds of formula (III), (IIIC) and (IIID) n is 0 to 2 (e.g. 1) or in compounds of formula (IIIE) n is 1 or 2.

In another aspect the invention provides a compound of formula (III) or a pharmaceutically acceptable salt or solvate thereof in which:
X is oxygen;
Y is CH;
Q¹ is A¹(CR⁶R⁷)ₙ;
Q³ is selected from the group consisting of C₁₋₅alkyl and C₁₋₂alkyl substituted by one to five fluorine atoms;
Q⁴ is C₁₋₆alkyl;
Q⁵ is selected from the group consisting of hydrogen, C₁₋₃alkyl, C₁₋₂alkyl substituted by one to five fluorine atoms, C₁₋₃alkylO₂C, halogen, and C₁₋₃alkylS;
A¹ is an unsubstituted 5- or 6-membered heteroaryl or an unsubstituted 6-membered aryl, or a 5- or 6-membered heteroaryl or a 6-membered aryl substituted by one or more R⁸;
Q⁸ is selected from the group consisting of halogen, C₁₋₆alkyl, C₁₋₆alkyl substituted by one more fluorine atoms, C₁₋₆alkoxy, and C₁₋₆alkoxy substituted by one or more F;
Q¹⁰ is selected from the group consisting of H and halogen; and
n is 0.

Compounds of formula (III) and salts and solvates thereof are described in PCT publication No. WO 2004/024691, published 25 Mar. 2004. The disclosures of these references are incorporated herein by reference in their entirety. Compounds of formula (III) may be prepared by any method described in WO 2004/024691 and equivalent patent applications.

In a further embodiment, the present invention provides compounds of formula (III) and pharmaceutically acceptable salts or solvates thereof for use in the preparation of a medicament for the treatment of depressive disorders as defined above.

In another embodiment, the present invention a method for the treatment of bipolar disorder, bipolar depression, bipolar disorder I, bipolar disorder II, unipolar depression comprising administering a therapeutically effective amount an effective amount of a first component which is a compound of formula (III) and pharmaceutically acceptable salts or solvates thereof, in combination with an effective amount of a second component which is a selective serotonin reuptake inhibitor.

In one embodiment of the present invention provides the use of a compound of formula selected from the following group consisting of:
2-(4-fluorophenoxy)-4-[4-(methylsulfonyl)phenyl]-6](trifluoromethyl)pyrimidine;
2-(4-methoxyphenoxy)-4-[4-(methylsulfonyl)phenyl]-6-trifluoromethyl)pyrimidine;
2-butoxy-4-[4-(methylsulfonyl)phenyl]-6-(trifluoromethyl)pyrimidine;
2-[(5-chloropyridin-3-yl)oxy]-4-[4-(methylsulfony)phenyl]-6-(trifluoromethyl)pyrimidine;
2-(cyclohexyloxy)-4-[4-(methylsulfonyl)phenyl]-6-(trifluoromethyl)pyrimidine;
3-(4-methylsulfonyl-phenyl)-2-(4-methoxy-phenyl)-pyrazolo[1,5-b]pyridazine;
6-difluoromethoxy-2-(4-fluoro-phenyl)-3-(4-methylsulfonyl-phenyl)-pyrazolo[1,5-b]-pyridazine;
2-(4-ethoxy-phenyl)-3-(4-methylsulfonyl-phenyl)-pyrazolo[1,5-b]pyridazine;
2-(4-fluoro-phenyl)-6-methylsulfonyl-3-(4-methylsulfonyl-phenyl)-pyrazolo[1,5-b]pyridazine;
2-(4-difluoromethoxy-phenyl)-3-(4-methylsulfonyl-phenyl)-pyrazolo[1,5-b]pyridazine;
4-[2-(4-ethoxy-phenyl)-pyrazolo[1,5-b]pyridazin-3-yl]-benzenesulfonamide;
6-difluoromethoxy-2-(3-fluoro-phenyl)-3-(4-methylsulfonyl-phenyl)-pyrazolo[1,5-b]pyridazine;
3-(4-methanesulfonyl-phenyl)-2-(4-methoxy-phenyl)-pyrazolo[1,5-b]pyridazine;
6-difluoromethoxy-2-(4-fluoro-phenyl)-3-(4-methanesulfonyl-phenyl)-pyrazolo[1,5-b]pyridazine;
2-(4-ethoxy-phenyl)-3-(4-methanesulfonyl-phenyl)-pyrazolo[1,5-b]pyridazine;
2-(4-fluoro-phenyl)-6-methanesulfonyl-3-(4-methanesulfonyl-phenyl)-pyrazolo[1,5-b]pyridazine;
2-(4-difluoromethoxy-phenyl)-3-(4-methanesulfonyl-phenyl)-pyrazolo[1,5-b]pyridazine;
4-[2-(4-ethoxy-phenyl)-pyrazolo[1,5-b]pyridazin-3-yl]-benzenesulfonamide;
6-difluoromethoxy-2-(3-fluoro-phenyl)-3-(4-methanesulfonyl-phenyl)-pyrazolo[1,5-b]pyridazine
4-ethyl-6-[4-(methylsulfonyl)phenyl]-N-(tetrahydro-2H-pyran-4-ylmethyl)-2-pyridinamine; 4-methyl-N-[(1-methyl-1H-pyrazol-4-yl)methyl]-6-[4-(methylsulfonyl)phenyl]-2-pyridinamine;
N-[(1,5-dimethyl-1H-pyrazol-4-yl)methyl]-4-methyl-6-[4-(methylsulfonyl)phenyl]-2-pyridinamine;
N-[(1,3-dimethyl-1H-pyrazol-4-yl)methyl]-4-methyl-6-[4-(methylsulfonyl)phenyl]-2-pyridinamine;
4-(6-{[(1,3-dimethyl-1H-pyrazol-4-yl)methyl]amino}-4-ethyl-2-pyridinyl)benzenesulfonamide;
N-[(1,3-dimethyl-1H-pyrazol-4-yl)methyl]-6-[4-(methylsulfonyl)phenyl]-4-(trifluoromethyl)-2-pyridinamine;
N-[(1,5-dimethyl-1H-pyrazol-4-yl)methyl]-6-[4-(methylsulfonyl)phenyl]-4-(trifluoromethyl)-2-pyridinamine;
4-{4-methyl-6-[(tetrahydro-2H-pyran-4-ylmethyl)amino]-2-pyridinyl}benzenesulfonamide;
4-methyl-N-[(1-methyl-1H-pyrazol-3-yl)methyl]-6-[4-(methylsulfonyl)phenyl]-2-pyridinamine;
N-(cyclohexylmethyl)-6-[4-(methylsulfonyl)phenyl]4-(trifluoromethyl)-2-pyrdinamine;
N-cyclohexyl-6-[4-(methylsulfonyl)phenyl]-4-(trifluoromethyl)-2-pyridinamine;
2-[4-(methylsulfonyl)phenyl]-6-[(2-pyridinylmethyl)oxy]-4-(trifluoromethyl)pyridine;
4-methyl-N-[(3-methyl-4-isoxazolyl)methyl]-6-[4-(methylsulfonyl)phenyl]-2-pyridinamine;
6-[4-(methylsulfonyl)phenyl]-N-(2-pyridinylmethyl)-4-(trifluoromethyl)-2-pyridinamine;
N-cycloheptyl-6-[4-(methylsulfonyl)phenyl]-4-(trifluoromethyl)-2-pyridinamine;
N-(cis-4methylcyclohexyl)-6-[4-(methylsulfonyl)phenyl]-4-(trifluoromethyl)-2-pyridinamine;
N-(1-ethylpropyl)-6-[4-(methylsulfonyl)phenyl]-4-(trifluoromethyl)-2-pyridinamine;
N-[(3-methyl-1,2,4-oxadiazol-5-yl)methyl]-6-[4-(methylsulfonyl)phenyl]-4-(trifluoromethyl)-2-pyridinamine;
N-[(5-methyl-1,2,4-oxadiazol-3-yl)methyl]-6-[4-(methylsulfonyl)phenyl]-4-(trifluoromethyl)-2-pyridinamine;
4-methyl-N-[(1-methyl-1H-pyrazol-5-yl)methyl]-6-[4-(methylsulfonyl)phenyl]-2-pyridinamine;
N-(cyclopentylmethyl)-6-[4-(methylsulfonyl)phenyl]4-(trifluoromethyl)-2-pyridinamine;
N-[(1-ethyl-1H-1,2,4-triazol-5-yl)methyl]-4-methyl-6-[4-(methylsulfonyl)phenyl]-2-pyridinamine;
4-ethyl-6-[4-(methylsulfonyl)phenyl]-2-[(2-pyridinylmethyl)amino]-3-pyridinecarbonitrile;
4-ethyl-2-{[(5-methyl-2-pyridinyl)methyl]amino}-6-[4-(methylsulfonyl)phenyl]-3-pyridinecarbonitrile;
4-ethyl-2-{[(6-methyl-3-pyridinyl)methyl]amino}-6-[4-(methylsulfonyl)phenyl]-3-pyridinecarbonitrile;
4-ethyl-2-{[(1-methyl-1H-pyrazol-4-yl)methyl]amino}-6-[4-(methylsulfonyl)phenyl]-3-pyridinecarbonitrile;
4-ethyl-6-[4-(methylsulfonyl)phenyl]-2-{[(4-methyl-1,3-thiazol-2-yl)methyl]amino}-3-pyridinecarbonitrile;
4-ethyl-6-[4-(methylsulfonyl)phenyl]-2-[(2-pyridinylmethyl)oxy]-3-pyridinecarbonitrile;
4-ethyl-N-[(1-ethyl-1H-1,2,4-triazol-5-yl)methyl]-6-[4-(methylsulfonyl)phenyl]-2-pyridinamine;
4-ethyl-2-{[(6-methyl-3-pyridinyl)methyl]oxy}-6-[4-(methylsulfonyl)phenyl]-3-pyridinecarbonitrile;
6-[4-(methylsulfonyl)phenyl]-N-[(1-methyl-1H-1,2,4-triazol-5-yl)methyl]-4-(trifluoromethyl)-2-pyridinamine;
   and pharmaceutically acceptable salts and solvates thereof, for use in the treatment of depressive disorders as defined above and the preparation of a medicament for the treatment of depressive disorders

In a particular embodiment of the present invention the compound is selected from the group consisting of: 2-(4-ethoxy-phenyl)-3-(4-methylsulfonyl-phenyl)-pyrazolo[1,5-b]pyridazine; 2-butoxy-4-[4-(methylsulfonyl)phenyl]-6-(trifluoromethyl)pyrimidine; N-cyclohexyl-6-[4-(methylsulfonyl)phenyl]-4-(trifluoromethyl)-2-pyridinamine; 2-[4-(methylsulfonyl)phenyl]-6-[(2-pyridinylmethyl)oxy]-4-(trifluoromethyl)pyridine; 4-methyl-N-[(1-methyl-1H-pyrazol-4-yl)methyl]-6-[4-(methylsulfonyl)phenyl]-2-pyridinamine; 3-(4-methanesulfonyl-phenyl)-2-(4-methoxy-phenyl)-pyrazolo[1,5-b]pyridazine; 6-difluoromethoxy-2-(4-fluoro-phenyl)-3-(4-methanesulfonyl-phenyl)-pyrazolo[1,5-b]pyridazine; 2-(4-ethoxy-phenyl)-3-(4-methanesulfonyl-phenyl)-pyrazolo[1,5-b]pyridazine; 2-(4-fluoro-phenyl)-6-methanesulfonyl-3-(4-methanesulfonyl-phenyl)-pyrazolo[1,5-b]pyridazine; 2-(4-difluoromethoxy-phenyl)-3-(4-methanesulfonyl-phenyl)-pyrazolo[1,5-b]pyridazine; 4-[2-(4-ethoxy-phenyl)-pyrazolo[1,5-b]pyridazin-3-yl]-benzenesulfonamide; 6-difluoromethoxy-2-(3-fluoro-phenyl)-3-(4-methanesulfonyl-phenyl)-pyrazolo[1,5-b]pyridazine; or a pharmaceutically acceptable salt or solvate thereof.

It is intended that reference to particular compounds herein be interpreted to mean that the pharmaceutically acceptable salts, solvates and prodrugs of those compounds may also be employed.

Conveniently, compounds of formula (I), (II) and (III) of the invention are isolated following work-up in the form of the free base. Pharmaceutically acceptable acid addition salts of the compounds of the invention may be prepared using conventional means.

Typically, a pharmaceutical acceptable salt may be readily prepared by using a desired acid or base as appropriate. The salt may precipitate from solution and be collected by filtration or may be recovered by evaporation of the solvent.

Suitable addition salts are formed from acids which form non-toxic salts and examples are hydrochloride, hydrobromide, hydroiodide, sulphate, bisulphate, nitrate, phosphate, hydrogen phosphate, acetate, maleate, malate, fumarate, lactate, tartrate, citrate, formate, gluconate, succinate, piruvate, oxalate, oxaloacetate, trifluoroacetate, saccharate, benzoate, methansulphonate, ethanesulphonate, benzenesulphonate, p-oluensulphonate, methanesulphonic, ethanesulphonic, p-toluenesulphonic, and isethionate.

In addition, prodrugs are also included within the context of this invention.

As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

Prodrugs are any covalently bonded carriers that release a compound of structure (I), (II) and (III) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol, sulfhydryl and amine functional groups of the compounds of structure (I).

With regard to stereoisomers, the compounds of structure (I), (II) and (III) may have one or more asymmetric carbon atom and may occur as recemates, racemic mixtures and as individual enantiomers or diastereomers. All such isomeric forms are included within the present invention, including mixtures thereof.

Similarly, when the invention is regarded in its broadest sense, the second component compound is a compound with anti-depressant activity.

In one aspect of the present invention the second component is a compound which functions as a selective serotonin reuptake inhibitor. The measurement of a compound's activity as an SSRI is now a standard pharmacological assay. Wong, et al., Neuropsychopharmacology 8, 337-344 (1993). Many compounds have such activity, and no doubt many more will be identified in the future. In the practice of the present invention, it is intended to include reuptake inhibitors which show 50% effective concentrations of about 1000 nM or less, in the protocol described by Wong supra.

Exemplary selective serotonin reuptake inhibitors include, but are not limited to: citalopram, escitalopram, fluoxetine, R-fluoxetine, sertraline, paroxetine, fluvoxamine, venlafaxine, duloxetine, dapoxetine, nefazodone, imipramine, imipramine N-oxide, desipramine, pirandamine, dazepinil, nefopam, befuraline, fezolamine, femoxetine, clomipramine, cianoimipramine, litoxetine, cericlamine, seproxetine, WY 27587, WY 27866, imeldine, ifoxetine, tiflucarbine, viqualine, milnacipran, bazinaprine, YM 922, S 33005, F 98214-TA, OPC 14523, alaproclate, cyanodothepine, trimipramine, quinupramine, dothiepin, amoxapine, nitroxazepine, McN 5652, McN 5707, VN 2222, L 792339, roxindole, YM 35992,0177, Org 6582, Org 6997, Org 6906, amitriptyline, amitriptyline N-oxide, nortriptyline, CL 255.663, pirlindole, indatraline, LY 113.821, LY 214.281, CGP 6085 A, RU 25.591, napamezole, diclofensine, trazodone, EMD 68.843, BMY 42.569, NS 2389, sercloremine, nitroquipazine, ademethionine, sibutramine, clovoxamine. The compounds mentioned above may be used in the form of the base or a pharmaceutically acceptable acid addition salt thereof.

In a further embodiment of the present invention the selective serotonin reuptake inhibitors of the present invention include, but are not limited to:

Fluoxetine, N-methyl-3-(p-trifluoromethylphenoxy)-3-phenylpropylamine, is marketed in the hydrochloride salt form, and as the racemic mixture of its two enantiomers. U.S. Pat. No. 4,314,081 is an early reference on the compound. Robertson et al., J. Med. Chem. 31, 1412 (1988), taught the separation of the R and S enantiomers of fluoxetine and showed that their activity as serotonin uptake inhibitors is similar to each other. In this document, the word “fluoxetine” will be used to mean any acid addition salt or the free base, and to include either the racemic mixture or either of the R and S enantiomers;

Duloxetine, N-methyl-3-(1-naphthalenyloxy)-3-(2-thienyl) propanamine, is usually administered as the hydrochloride salt and as the (+) enantiomer. It was first taught by U.S. Pat. No. 4,956,388, which shows its high potency. The word “duloxetine” will be used here to refer to any acid addition salt or the free base of the molecule;

Venlafaxine is known in the literature, and its method of synthesis and its activity as an inhibitor of serotonin and norepinephrine uptake are taught by U.S. Pat. No. 4,761,501. Venlafaxine is identified as compound A in that patent;

Milnacipran (N,N-diethyl-2-aminomethyl-1-phenylcyclopropanecarboxamide) is taught by U.S. Pat. No. 4,478,836, which prepared milnacipran as its Example 4. The patent describes its compounds as antidepressants. Moret et al., Neuropharmacology 24, 1211-19 (1985), describe its pharmacological activities as an inhibitor of serotonin and norepinephrine reuptake;

Citalopram, 1-[3-(dimethylamino)propyl]-1-(4-fluorophenyl)-1,3-dihydro-5-isobenzofuran-aqscarbonitrile, is disclosed in U.S. Pat. No. 4,136,193 as a serotonin reuptake inhibitor. Its pharmacology was disclosed by Christensen et al., Eur. J. Pharmacol. 41, 153 (1977), and reports of its clinical effectiveness in depression may be found in Dufour et al., Int. Clin. Psychopharmacol. 2, 225 (1987), and Timmerman et al., ibid., 239;

Fluvoxamine, 5-methoxy-1-[4-(trifluoromethyl)-phenyl]-1-pentanone-O-(2-aminoethyl)oxime, is taught by U.S. Pat. No. 4,085,225. Scientific articles about the drug have been published by Claassen et al., Brit. J. Pharmacol. 60, 505 (1977); and De Wilde et al., J. Affective Disord. 4, 249 (1982); and Benfield et al., Drugs 32, 313 (1986);

Paroxetine, trans-(−)-3-[(1,3-benzodioxol-5-yloxy)methyl]-4-(4-fluorophenyl)piperidine, may be found in U.S. Pat. Nos. 3,912,743 and 4,007,196. Reports of the drug's activity are in Lassen, Eur. J. Pharmacol. 47, 351 (1978); Hassan et al., Brit J. Clin. Pharmacol. 19, 705 (1985); Laursen et al., Acta Psychiat. Scand. 71, 249 (1985); and Battegay et al., Neuropsychobiology 13, 31 (1985);

Sertraline, (1S-cis)-4-(3,4-dichlorophenyl)-1,2,3,4-tetrahydro-N-methyl-1-naphthylamine hydrochloride, is a serotonin reuptake inhibitor which is marketed as an antidepressant. It is disclosed by U.S. Pat. No. 4,536,518;

All of the U.S. patents which have been mentioned above in connection with compounds used in the present invention are incorporated herein by reference.

In one aspect of the present invention it will be understood that while the use of a single COX-2 inhibitor of the present invention as a first component compound is preferred, combinations of two or more COX-2 inhibitors of the present invention may be used as a first component if necessary or desired. Similarly, while the use of a single selective serotonin reuptake inhibitor as a second component compound is preferred, combinations of two or more serotonin reuptake inhibitors may be used as a second component if necessary or desired.

Combinations can also include a mixture of one or more COX-2 inhibitors of the present invention or a mixture of one COX-2 inhibitor of the present invention with another COX-2 inhibitor, for example, available on the market (Celebrex®).

In a further special embodiment of the present invention combinations of first and second component compounds are selected in the following group:
first component: 2-(4-ethoxy-phenyl)-3-(4-methylsulfonyl-phenyl)-pyrazolo[1,5-b]pyridazine;
2-butoxy4-[4-(methylsulfonyl)phenyl]-6-(trifluoromethyl)pyrimidine;
N-cyclohexyl-6-[4-(methylsulfonyl)phenyl]-4-(trifluoromethyl)-2-pyrdinamine;
4-[2-(4-ethoxy-phenyl)-pyrazolo[1,5-b]pyridazin-3-yl]-benzenesulfonamide;
a pharmaceutically acceptable salt of N-cyclohexyl-6-[4-(methylsulfonyl)phenyl]-4-(trifluoromethyl)-2-pyridinamine;
a pharmaceutically acceptable salt of 2-butoxy-4-[4-(methylsulfonyl)phenyl]-6-(trifluoromethyl)pyrimidine; N-cyclohexyl-6-[4-(methylsulfonyl)phenyl]-4-(trifluoromethyl)-2-pyridinamine;
a pharmaceutically acceptable salt of N-cyclohexyl-6-[4-(methylsulfonyl)phenyl]-4-(trifluoromethyl)-2-pyridinamine;
a pharmaceutically acceptable salt of 4-[2-(4-ethoxy-phenyl)-pyrazolo[1,5-b]pyridazin-3-yl]-benzenesulfonamide;
second component: paroxetine.

It will be understood by the skilled reader that most or all of the compounds used in the present invention are capable of forming salts, and that the salt forms of pharmaceuticals are commonly used, often because they are more readily crystallized and purified than are the free bases. In all cases, the use of the pharmaceuticals described above as salts is contemplated in the description herein, and often is preferred, and the pharmaceutically acceptable salts of all of the compounds are included in the names of them.

The dosages of the drugs used in the present invention must, in the final analysis, be set by the physician in charge of the case, using knowledge of the drugs, the properties of the drugs in combination as determined in clinical trials, and the characteristics of the patient, including diseases other than that for which the physician is treating the patient. General outlines of the dosages, and some preferred dosages, can and will be provided here.

Dosage guidelines for some of the drugs will first be given separately; in order to create a guideline for any desired combination, one would choose the guidelines for each of the component drugs.
Fluoxetine: from about 1 to about 80 mg, once/day; preferred, from about 10 to about 40 mg once/day; preferred for bulimia and obsessive-compulsive disease, from about 20 to about 80 mg once/day;
Duloxetine: from about 1 to about 160 mg once/day; or up to 80 mg twice daily; preferred, from about 5 to about 20 mg once/day;
Venlafaxine: from about 10 to about 150 mg once-thrice/day; preferred, from about 25 to about 125 mg thrice/day;
Milnacipran: from about 10 to about 100 mg once-twice/day; preferred, from about 25 to about 50 mg twice/day;
Citalopram: from about 5 to about 50 mg once/day; preferred, from about 10 to about 30 mg once/day;
Fluvoxamine: from about 20 to about 500 mg once/day; preferred, from about 50 to about 300 mg once/day;
Paroxetine: from about 20 to about 50 mg once/day; preferred, from about 20 to about 30 mg once/day;
Sertraline: from about 20 to about 500 mg once/day; preferred, from about 50 to about 200 mg once/day;

In another aspect the present invention provides alternatives to the selective serotonin reuptake inhibitors as second component to be combined with the compounds of formula (I) (II) and (III) as first component.

Various types of antidepressants can be used as second component according to the present invention. Examples of antidepressants that are useful in the present invention include, but are not limited to:
tricyclic antidepressants such as amitriptyline (5-(3-dimethylamino propylidene)-10,11-dihydro-5H-dibenzo[a,d]cyclohepten), amitriptyline oxide, desipramine (10,11-dihydro-5-(3-methylaminopropyl)-5H-dibenz[b,f]flazepin), dibenzepin (10-(2-dimethylaminoethyl)-5,11-dihydro-5-methyl11H-dibenzo[b,e][1,4]diazepin-11-on), dosulepin (3-(6H-dibenzo[b,e]thiepin-11-yliden)-N,N-dimethylpropylamine), doxepin (3-(6H-dibenzo[b,e]oxepin-11-yliden)-dimethylpropylamine), chloroimipramine, imipramine (5-(3-dimethylaminopropyl)-5,11-dihydro-5H-dibenz[b,f]azepin), nortriptyline (3-(10,11-dihydro-5H-dibenzo[a,d]cyclohepten-5-yliden)-N-methyl-1-propaneamine), mianserin (1, 2, 3, 4, 10, 14b-hexahydro-2-methyl-dibenzo[c,f]pyrazino[1,2-a]azepin), maprotiline (N-methyl-9,10-ethanoanthracene-9(10H)-propaneamine), trimipramine (5-[3-dimethylamino)-2-methylpropyl]-10,11-dihydro-5H-dibenz[b,f]azepin) or viloxazine (RS)-2-(2-ethyoxyphenoxymethyl)-morpholine),
modern antidepressants such as trazodone (2-{3-[4-(3-chlorophenyl)-1-piperazinyl]-propyl}-1,2,4-triazol[4,3a]pyridine-3(2H)-on, nefazodone (2-{3-[4-(3-chlorophenyl)-1-piperazinyl]propyl)-5-ethyl-2,4-dihydro-4-(2-phenoxyethyl)-3H-1,2,4-triazol-3-on), mirtazapine ((±)-1,2,3,4,10,14b-hexahydro-2-methylpyrazino[2,1-a] pyrido[2,3-c][2] benzazepine), bupropion, (+/−)-(1-(3-chlorophenyl)-2-((1,1 -dimethylethyl)amino)-1-propanone, venlafaxine ( ( )1-2-(dimethylamino)-1-(4-methoxyphenyl)-ethyl] cyclohexanol) or reboxetine ((±)-(2RS)-2-[(αSR)-α-(2-ethoxyphenoxy)benzyl] morpholine), inhibitors of monoaminooxidases such as tranylcypromine (trans-2-phenyl cyclopropylamine), brofaromine or moclobemide (4-chloro-N-(2-morpholinoethyl)-benzamide), and vegetable antidepressants such as Hypericum (St. John's wort).

Selective antagonists of NK₁ receptor for use in the present invention as second component include those generically and specifically disclosed in the following patent specifications whose disclosures are here incorporated by reference:

US Patent Specification Nos. 4839465, 5338845, 5594022, 6169097, 6197772, 6222038, 6204265, 6329392, 6316445, 2001039286, 2001034343, 2001029297, 2002193402, 2002147212, 2002147207, 2002143003 and 2002022624; and in European Patent Specification Nos. 284942, 327009, 333174, 336230, 360390, 394989, 428434, 429366, 436334, 443132, 446706, 482539, 484719, 499313, 512901, 512902, 514273, 514275, 517589, 520555, 522808, 525360, 528495, 532456, 533280, 577394, 591040, 615751, 684257, 1176144, 1110958, 1176144, 1172106, 1103545, and 1256578; and in International Patent Application Nos. 90/05525, 90/05729, 91/02745, 91/12266, 91/18016, 91/18899, 92/01688, 92/06079, 92/15585, 92/17449, 92/20676, 92/21677, 92/22569, 93/00331, 93/01159, 93/01160, 93/01165, 93/01169, 93/01170, 94/01402, 94/26735, 95/06645, 95/08549, 95/14017, 95/16679, 95/18124, 95/23798, 95/28389, 95/33744, 96/05181, 96/18643, 96/21661, 96/29326, 96/32386, 96/34857, 96/37489, 97/02824, 97/05110, 97/08166, 97/13514, 97/14671, 97/16440, 97/17362, 97/19074, 97/19084, 97/19942, 97/21702, 97/22597, 97/22604, 97/23455, 97/24324, 97/24350, 97/25322, 97/25988, 97/27185, 97/30989, 97/30990, 97/30991, 97/32865, 97/38692, 97/44035, 97/49393, 97/49710, 98/02158, 98/04561, 98/07694, 98/07722, 98/08826, 98/13369, 98/17276, 98/18761, 98/18785, 98/18788, 98/20010, 98/24438, 98/24439, 98/24440, 98/24441, 98/24442, 98/24442, 98/24443, 98/24444, 98/24445, 98/24446, 98/24447, 98/28297, 98/43639, 98/45262, 98/49170, 98/54187, 98/57954, 98/57972, 99/00388, 99/01444, 99/01451, 99/07677, 99/07681, 99/09987, 99/21823, 99/24423, 99/25364, 99/26924, 99/27938, 99/36424, 99/52903, 99/59583, 99/59972, 99/62893, 99/62900, 99/64000, 00/02859, 00/06544, 00/06571, 00/06572, 00/06578, 00/06580, 00/15621, 00/20003, 00/21512, 00/21564, 00/23061, 00/23062, 00/23066, 00/23072, 00/20389, 00/25745, 00/26214, 00/26215, 00/34243, 00/34274, 00/39114, 00/47562, 01/77069, 01/25233, 01/30348, 01/87866, 01/94346, 01/90083, 01/87838, 01/85732, 01/77100, 01/77089, 01/77069, 01/46176, 01/46167, 01/44200, 01/32625, 01/29027, 01/25219, 02/32865, 02/00631, 02/81461, 02/92604, 02/38575, 02/57250, 02/22574, 02/74771, 02/26710, 02/28853, 02/102372, 02/85458, 02/81457, 02/74771, 02/62784, 02/60898, 02/60875, 02/51848, 02/51807, 02/42280, 02/34699, 02/32867, 02/32866, 02/26724, 02/24673, 02/24629, 02/18346, 02/16344, 02/16343, 02/16324, 02/12168, 02/08232 and 02/06236; and in British Patent Specification Nos. 2216529, 2266529, 2268931, 2269170, 2269590, 2271774, 2292144, 2293168, 2293169 and 2302689; and in Japanese Patent Specification No 6040995. A special useful class of NK1 receptor antagonists for use in the combinations of the present invention is represented by those compounds described in WO 01/25219. In a further embodiment of the present invention the compound 2-(S)-(4-fluoro-2-methyl-phenyl)-piperazine-1-carboxylic acid [1-(R)-(3,5-bis-trifluoromethyl-phenyl)-ethyl]-methyl-amide methansulphonate may be used.

Selective antagonists of CRF-1 receptor for use in the present invention as second component include those generically and specifically disclosed in the following patent specifications whose disclosures are here incorporated by reference:

US Patent Specification Nos.: 4,605,642, 5,063,245, 6,348,466, 6,348,466 and in International Patent Application Nos. 94/13676, 94/13677, 95/10506, 95/33727, 95/33750, 95/34563, 96/35689, 96/39400, 97/00868, 97/14684, 97/29109, 97/29110, 97/35580, 97/35846, 97/44038, 98/03510, 98/05661, 98/08821, 98/08846, 98/08847, 98/11075, 98/15543, 98/21200, 98/27066, 98/29397, 98/29413, 98/35967, 98/42699, 98/45295, 98/47874, 98/47903, 99/01454, 99/01439, 99/00373, 99/10350, 99/12908, 99/38868, 00/27846, 00/27850, 01/44207, 02/87573, 02108895, 02/100863, 02/094826, 03/008412, 03/008414 and in European patent publications: 778277, 773023, 576350, 112909.

Other antidepressant drugs are disclosed in WO099/37305 and among them, (+)-(2S,3S)-2-(3-chlorophenyl)-3,5,5-trimethyl-2-morpholinol may be used in the present invention as second component.

In more general terms, one would create a combination of the present invention by choosing a dosage of first and second component compounds according to the spirit of the above guideline.

The adjunctive therapy of the present invention is carried out by administering a first component together with the second component in any manner which provides effective levels of the compounds in the body at the same time. All of the compounds concerned are orally available and are normally administered orally, and so oral administration of the adjunctive combination is preferred. They may be administered together, in a single dosage form, or may be administered separately.

However, oral administration is not the only route or even the only preferred route. For example, transdermal administration may be very desirable for patients who are forgetful or petulant about taking oral medicine. One of the drugs may be administered by one route, such as oral, and the others may be administered by the transdermal, percutaneous, intravenous, intramuscular, intranasal or intrarectal route, in particular circumstances. The route of administration may be varied in any way, limited by the physical properties of the drugs and the convenience of the patient and the caregiver.

The adjunctive combination may be administered as a single pharmaceutical composition, and so pharmaceutical compositions incorporating both compounds are important embodiments of the present invention. Such compositions may take any physical form which is pharmaceutically acceptable, but orally usable pharmaceutical compositions are particularly preferred. Such adjunctive pharmaceutical compositions contain an effective amount of each of the compounds, which effective amount is related to the daily dose of the compounds to be administered. Each adjunctive dosage unit may contain the daily doses of all compounds, or may contain a fraction of the daily doses, such as one-third of the doses. Alternatively, each dosage unit may contain the entire dose of one of the compounds, and a fraction of the dose of the other compounds. In such case, the patient would daily take one of the combination dosage units, and one or more units containing only the other compounds. The amounts of each drug to be contained in each dosage unit depends on the identity of the drugs chosen for the therapy, and other factors such as the indication for which the adjunctive therapy is being given.

The inert ingredients and manner of formulation of the adjunctive pharmaceutical compositions are conventional, except for the presence of the combination of the present invention. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches and suspensions. In general, compositions contain from about 0.5% to about 50% of the compounds in total, depending on the desired doses and the type of composition to be used. The amount of the compounds, however, is best defined as the effective amount, that is, the amount of each compound which provides the desired dose to the patient in need of such treatment. The activity of the adjunctive combinations do not depend on the nature of the composition, so the compositions are chosen and formulated solely for convenience and economy. Any of the combinations may be formulated in any desired form of composition. Some discussion of different compositions will be provided, followed by some typical formulations.

Capsules are prepared by mixing the compound with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours and similar edible powders.

Tablets are prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant is necessary in a tablet formulation to prevent the tablet and punches from sticking in the die. The lubricant is chosen from such slippery solids as talc, magnesium and calcium stearate, stearic acid and hydrogenated vegetable oils.

Tablet disintegrators are substances which swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethylcellulose, for example, may be used, as well as sodium lauryl sulfate.

Enteric formulations are often used to protect an active ingredient from the strongly acid contents of the stomach. Such formulations are created by coating a solid dosage form with a film of a polymer which is insoluble in acid environments, and soluble in basic environments. Exemplary films are cellulose acetate phthalate, polyvinyl acetate phthalate, hydroxypropyl methylcellulose phthalate and hydroxypropyl methylcellulose acetate succinate. It is preferred to formulate duloxetine and duloxetine-containing combinations as enteric compositions, and even more preferred to formulate them as enteric pellets.

Tablets are often coated with sugar as a flavor and sealant. The compounds may also be formulated as chewable tablets, by using large amounts of pleasant-tasting substances such as mannitol in the formulation, as is now well-established practice. Instantly dissolving tablet-like formulations are also now frequently used to assure that the patient consumes the dosage form, and to avoid the difficulty in swallowing solid objects that bothers some patients.

When it is desired to administer the combination as a suppository, the usual bases may be used. Cocoa butter is a traditional suppository base, which may be modified by addition of waxes to raise its melting point slightly. Water-miscible suppository bases comprising, particularly, polyethylene glycols of various molecular weights are in wide use, also.

Transdermal patches have become popular recently. Typically they comprise a resinous composition in which the drugs will dissolve, or partially dissolve, which is held in contact with the skin by a film which protects the composition. Many patents have appeared in the field recently. Other, more complicated patch compositions are also in use, particularly those having a membrane pierced with innumerable pores through which the drugs are pumped by osmotic action.

### EXAMPLE 1

### Preparation of Compounds of Formula (I)

Compounds of formula (I) may be prepared by any method described in WO 02/096885, U.S. application Ser. No. 10/477,547 and equivalent patent applications.

Intermediate 1

### 4,4,4-Trifluoro-1-[4-(methylthio)phenyl]butane-1,3-dione

To a solution of ethyl trifluoroacetate (7.95 ml, 1.1 eq) in MTBE (125 ml) was added dropwise 25% sodium methoxide in methanol (16 ml, 1.2 eq). 4-Methylthioacetophenone (Aldrich, 10 g, 0.06 mol) was added portionwise and the mixture stirred at ambient temperature overnight. 2N Hydrochloric acid (40 ml) was added cautiously and the organic phase separated, washed with brine and dried (Na₂SO₄) to give an orange solid. The orange solid was recrystallised from hot isopropanol to give the title compound as a yellow crystalline solid (11.25 g, 71%).

MH− 261

Intermediate 2

### 2-(Methylthio)-4-[4-(methylthio)phenyl]-6-(trifluoromethyl) pyrimidine

To a mixture of 4,4,4-trifluoro-1-[4-(methylthio)phenyl]butane-1,3-dione (5 g) and 2-methyl-2-thiopseudourea sulfate (5.1 g, 0.98 eq) in acetic acid (100 ml) was added sodium acetate (3 g, 2 eq) and heated under reflux for 8 h. The mixture was concentrated in vacuo and water (100 ml) added to give a solid, which was isolated by filtration to give the title compound as a yellow solid (5.8 g, quantitative).

MH+ 317

Intermediate 3

### 2-(Methylsulfonyl)-4-[4-(methylsulfonyl)phenyl]-6-(trifluoromethyl)pyrimidine

To a solution of 2-(methylthio)-4-[4-(methylthio)phenyl]-6-(trifluoromethyl) pyrimidine (5.78 g) in MeOH (500 ml) was added a solution of OXONE™ (Aldrich, 56.23 g, 5 eq) in water (200 ml). The mixture was stirred at ambient temperature overnight, concentrated in vacuo and the residue partitioned between water and ethyl acetate (2×100 ml). The combined organic phases were dried and concentrated in vacuo to an off-white solid which was triturated with hot isopropanol to give the title compound as a white solid (5.6 g, 80%).

MH+ 381

Tlc SiO₂ Ethyl acetate:cyclohexane (1:1) Rf 0.45

### EXAMPLE 1.1

### 2-(4-Fluorophenoxy)-4-[4-(methylsulfonyl)phenyl]-6-(trifluoromethyl)pyrimidine.

To a stirred solution of 4-fluorophenol (37 mg, 0.33 mmole) in dry tetrahyrofuran (10 ml) was added, under an atmosphere of nitrogen, sodium hydride (60% dispersion in oil, 13 mg, 0.33 mmole) and the resulting mixture stirred at 20 for 30 min. To the stirred reaction mixture was added 2-(methylsulfonyl)-4[4-(methylsulfonyl)phenyl]-6-trifluoromethyl)pyrimidine (114 mg, 0.33 mmole) in a single portion, and stirring was continued for 2 h. The solvent was evaporated, and the residue partitioned between dichloromethane and 2N sodium hydroxide. The dried organic phase was evaporated to dryness. The residue was purified on a silica gel SPE cartridge eluting with chloroform to afford the title compound as a colourless solid (99 mg, 80%).

MH+ 413.

### EXAMPLES 1.2 TO 1.10

Examples 1.2 to 1.10, as shown in Table 1 that follows, were prepared in the manner described for Example 1.1

TABLE 1

| | |
|---|---|
| | (I) |
| 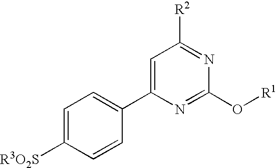 | |

| Ex | R¹ | R² | R³ | MS |
|---|---|---|---|---|
| 1.2 | 3,4-difluorophenyl | CF₃ | CH₃ | MH+ 431 |
| 1.3 | 4-methoxyphenyl | CF₃ | CH₃ | MH+ 425 |
| 1.4 | 4-fluorobenzyl | CF₃ | CH₃ | MH+ 427 |
| 1.5 | 4-bromophenyl | CF₃ | CH₃ | MH+ 474 |
| 1.6 | 4-methylphenyl | CF₃ | CH₃ | MH+ 409 |
| 1.7 | 5-chloropyridin-3-yl | CF₃ | CH₃ | MH+ 431 |
| 1.8 | cyclohexyl | CF₃ | CH₃ | MH+ 401 |
| 1.9 | cyclopentylmethyl | CF₃ | CH₃ | MH+ 401 |
| 1.10 | n-butyl | CF₃ | CH₃ | MH+ 375 |

### EXAMPLE 1.11

### 2-Butoxy-4-[4-(methylsulfonyl)phenyl]-6-(trifluoromethyl)pyrimidine

Sodium methoxide (6.6 kg of a 30% w/w solution in methanol) was added over at least 30 min to a solution of 4-(methylthio)acetophenone (5.0 kg) and methyl trifluoroacetate (4.25 kg) in tert-butylmethylether (40 L) at 40±3° C. The solution was heated at 40±3° C. for at least 3 h. Acetic acid (55 L) was added, followed by S-methyl 2-thiopseudourea sulfate (5.45 kg) and the mixture concentrated to ca. 45 L. The mixture was heated at about 110° C. for at least a further 8 h (overnight) then acetic acid (20 L) was added before cooling to 50±3° C. A solution of sodium tungstate dihydrate (0.2 kg) in water (2.5 L) was added, followed by hydrogen peroxide (20.7 kg of 30% w/v solution), which was added over at least 3 h, maintaining the temp at ca. 50°. The mixture is heated at ca. 50° C. for at least 12 h before cooling to 20±3° C. A solution of sodium sulphite (3.45 kg) in water (28 L) was then added over at least 30 min whilst maintaining the temperature at 20±3°. The mixture was aged at 20±3° C. for ca. 1 h and 2-(methylsulfonyl)-4-[4-(methylsulfonyl)phenyl]-6-(trifluoromethyl)pyrimidine_collected by filtration, washed with water (3×15 L) and dried at up to 60° in vacuo. Yield, 9.96 kg, 90% of theory.

A suspension of 2-(methylsulfonyl)-4-[4-(methylsulfonyl)phenyl]-6-(trifluoromethyl)pyrimidine (525 g) in n-butanol (5.25 L) was treated with potassium carbonate (210 g) at 20±5° C. The mixture was heated to 50±5° C. overnight until the reaction was complete by HPLC. Acetic acid (1.57 L) was added dropwise, to control any gas evolution, keeping the temperature at 50±5° C. Water (3.67 L) was then added over 30 min keeping the temperature at 50±5° C. to allow full crystallisation to occur. The slurry was then cooled to 20-25° C. and aged for at least 1 hour. The resulting product was then filtered under vacuum and washed with a mixture of n-butanol (787 mL), acetic acid (236 mL), and water (551 mL) followed by water (2×1.57 L). The product was then dried at up to ca50° C. under vacuum to yield the title compound. Yield, 457 g, 88.4% of theory. The title compound was found to be identical to that of Example 10.

¹H NMR (CDCl₃) δ: 8.33(2H, d, para-di-substituted CH); 8.11(2H, d, para-di-substituted CH); 7.70(1H, s, aromatic CH); 4.54(2H, t, butyl CH₂); 3.12(3H, s, sulphone CH₃); 1.88(2H, m, butyl CH₂); 1.55(2H, m, butyl CH₂); 1.01(3H, t, butyl CH₃).

### EXAMPLE 2

### Preparation of Compounds of Formula (II)

Compounds of formula (II) may be prepared by any method described in WO 99/12930, U.S. Pat. No. 6,451,794, US-A-2003-0040517 and US-A-2003-0008872 and equivalent patent applications.

### EXAMPLE 2.1

### 6-Difluoromethoxy-2-(4-fluoro-phenyl)-3-(4-methanesulfonyl-phenyl)-pyrazolo[5-b]pyridazine

(i) 6-Methoxy-2-(4-fluoro-phenyl-pyrazolo[1,5-b]pyridazine-3-carboxylic acid methyl ester.

1,8-Diazabicyclo[5.4.0]undec-7-ene (3.39 mL) was added to a mixture of 3-(4-fluorophenyl)-prop-2-ynoic acid methyl ester (3.36 g) and 1-amino-3-methoxy-pyridazin-1-ium mesitylene sulphonate¹ (6.1419 g) in acetonitrile (125 mL) and the mixture was stirred at ambient temperature for 48 hours. During the first 2 hours a stream of air was passed through the reaction. The mixture was concentrated in vacuo, dissolved in ethyl acetate (150 mL), washed with water (3×25 mL), dried (MgSO₄), filtered and evaporated in vacuo to give the title compound as a brown solid (4.77 g).

¹H NMR (CDCl₃): 8.4 (d, 1H, J=10 Hz) 7.85-7.90 (m, 2H) 7.1-7.2 (m, 2H) 6.9-7.0 (d, 1H, J=10 Hz) 4.1 (s, 3H) 3.9 (s, 3H)

MH⁺ 302

Ref:¹ T. Tsuchiya, J. Kurita and K. Takayama, Chem. Pharm. Bull. 28(9) 2676-2681 (1980).

(ii) 6-Methoxy-2-(4-fluoro-phenyl-pyrazolo[1,5-b]pyridazine-3-carboxylic acid

A mixture of 6-methoxy-2-(4-fluoro-phenyl-pyrazolo[1,5-b]pyridazine-3-carboxylic acid methyl ester (4.469 g), 2N sodium hydroxide (50 mL) and methanol (90 mL) was heated at reflux for 2 hours. The cooled solution was added to 2N hydrochloric acid (200 mL) and the title compound was isolated by filtration as a beige solid (3.639 g).

¹H NMR (DMSO-d₆): 12.8 (br. s, 1H) 8.4 (d, 1H, J=10 Hz) 7.8-7.9 (m, 2H) 7.21-7.32 (m, 2H) 7.15-7.2 (d, 1H, J=10 Hz) 4.0 (s, 3H)

MH⁺ 288

(iii) 2-(4-Fluoro-phenyl)-3-(4-methanesulfonyl-phenyl)-6-methoxy-pyrazolo[1.5-b]pyridazine

A mixture of 6-methoxy-2-(4-fluoro-phenyl-pyrazolo[1,5-b]pyridazine-3-carboxylic acid (869 mg) and sodium bicarbonate (756 mg) in dimethylformamide (10 mL) was treated with N-bromosuccinimide (587 mg) and stirred at ambient temperature for 1 hour, then added to water (50 mL) and extracted with ethyl acetate (3×50 mL), dried (MgSO₄), and evaporated in vacuo. The resulting brown solid (1.612 g) was dissolved in 1,2 dimethoxyethane (20 mL). 2N Aqueous sodium carbonate solution (10 mL) was added together with 4-(methanesulphonyl)phenyl boronic acid (660 mg) and tetrakis(triphenylphosphine)palladium (O) (100 mg) and the mixture was heated at reflux for 20 hours. The reaction was poured into water (50 mL), extracted with dichloromethane (3×100 mL). The combined organic extracts were dried (MgSO₄) and evaporated in vacuo to give a brown solid (1.116 g) which was purified by flash column chromatography on silica, eluting with cyclohexane/ethyl acetate (4:1 then 2:1), to give the title compound as a yellow solid (390 mg).

Tlc, SiO₂, R_{f} 0.3 (1:1 cyclohexane/ethyl acetate), detection UV

MH⁺ 398

(iv) 2-(4-Fluoro-phenyl)-3-(4-methanesulfonyl-phenyl)-pyrazolo[1,5-b]pyridazin-6-ol

A mixture of 2-(4-fluoro-phenyl)-3-(4-methanesulfonyl-phenyl)-6-methoxy-pyrazolo[1,5-b]pyridazine (321 mg) and pyridine hydrochloride (1.4 g) was heated to and at 200° C. in a sealed vessel (Reactivial™) for 3 hours. The cooled reaction was poured into water (20 mL), and extracted with ethyl acetate (3×30 mL). The combined organic extracts dried (MgSO₄), filtered and evaporated in vacuo to give a solid which was triturated with diethyl ether to give the title compound as a beige solid (119 mg).

Tlc, SiO₂, Rf 0.07 (1:2 cyclohexane/ethyl acetate), detection UV.

MH⁺ 384

(v) 6-Difluoromethoxy-2-(4-fluoro-phenyl)-3-(4-methanesulfonyl-phenyl)-pyrazolo[1,5-b]pyridazine

A solution of 2-(4-fluoro-phenyl)-3-(4-methanesulfonyl-phenyl)-pyrazolo[1,5-b]pyridazin-6-ol (0.2 g) in anhydrous dimethyl formamide (5 mL) was treated with sodium hydride ( 0.046 g, 60% dispersion in mineral oil), after effervescence ceased a stream of bromodifluoromethane gas was passed through the mixture at ambient temperature for 30 minutes. The reaction mixture was then poured into water (50 mL) and extracted with ethyl acetate (50 mL), the organic extract was washed with water (3×50 mL), dried and concentrated in vacuo. The residue was purified by chromatography to give the title compound as a white solid (0.17 g).

MH⁺=434

¹HNMR(CDCl₃):δ8.05-8.0(d,J=10HZ,2H)8.0-7.95(d,J=10HZ,1H)7.6-7.5(m,4H)7.8-7.2(t,J=70HZ,1H)7.1-7.05(t,J=11HZ,2H)6.9-6.85(d,J=10HZ,1H)3.15(s,3H)

Tlc,SiO₂,Rf 0.35(ethyl acetate/cyclohexane(1/1))

### EXAMPLE 2.2

### 3-(4-Methanesulfonyl-phenyl)-2-(4-methoxy-phenyl)-pyrazolo[1,5-b]pyridazine

(i) 2-(4-Methoxy-phenyl)-pyrazolo[1,5-b]pyridazine-3-carboxylic acid methyl ester

Diazabicyclo[5.4.0]undec-7-ene (22.76 mL, 2 eq) was added dropwise to a solution of methyl 3-(4-methoxy-phenyl)-prop-2-ynoic acid¹ (14.46 g, 76 mM) and 1-amino pyridazinium iodide² (2 eq) in acetonitrile under nitrogen and stirred for 6 h. Purification by chromatography on silica gel eluting with toluene, then toluene:ethyl acetate (9:1) gave the title compound (2.769) as a brown solid.

MH⁺ 284

1H NMR (CDCl₃) δ 3.87 (3H, s) 3.9 (3H, s) 7.0 (2H, d, J=9 Hz) 7.25 (1H, dd, J=9 & 4 Hz) 7.90 (2H, d, J=9 Hz) 8.45 (1H, dd, J=4 & 2 Hz) 8.55 (1H, dd, J=9 & 2 Hz)

Ref:¹ J. Morris and D. G. Wishka, Synthesis (1994), (1), 43-6

Ref:² Kobayashi et al Chem. Pharm. Bull. (1971), 19 (10), 2106-15

(ii) 3-(4-Methanesulfonyl-phenyl)-2-(4-methoxy-phenyl)-pyrazolo[1,5-b]pyridazine

A mixture of 2-(4-methoxy-phenyl)-pyrazolo[1,5-b]pyridazine-3-carboxylic acid methyl ester (2.76 g) and aq. sodium hydroxide (2N, 30 mL) in ethanol (30 mL) was refluxed under nitrogen for 2 h. The cooled mixture was acidified with hydrochloric acid (2N) and the resulting white solid (2.53 g) isolated by filtration. This solid was dissolved in DMF and sodium bicarbonate (2.67 g, 3.3 eq) added, followed by N-bromosuccinimide (1.88 g, 1.1 eq) portionwise. After stirring for 1 h under nitrogen, water was added and extracted into ethyl acetate (2×25 mL). The dried organic phase was concentrated and the residue taken up in DME (60 mL). Aqueous sodium carbonate (2N, 15 mL) was added, followed by 4-methanesulfonyl-phenylboronic acid (3.12 g) and tetrakis(triphenylphosphine)palladium(O) (250 mg). The mixture was heated at reflux under nitrogen for 18 h, cooled, poured into water and extracted into ethyl acetate (2×25 mL). The combined organic phases were dried and concentrated onto silica gel. Chromatography on silica gel eluting with toluene:ethyl acetate (8:1) gave, on concentration, the title compound (3.58 g) as a cream solid.

MH⁺ 380

1H NMR (DMSO) δ 3.25 (3H, s) 3.75 (3H, s) 6.95 (2H, d, J=8.5 Hz) 7.25 (1H, dd, J=9 & 5 Hz) 7.45 (2H, d, J=8.5 Hz) 7.60 (2H, d, J=8Hz) 7.9 (2H, d, J=8.5 Hz) 8.15 (1H, dd, J=9 & 2 Hz) 8.49 (1H, dd, J=5 & 2 Hz)

### EXAMPLE 2.3

### 2-(4-Ethoxy-phenyl)-3-(3-methanesulfonyl-phenyl)-pyrazolo[1,5-b]pyridazine

(i) 4-[3-(4-Methanesulfonyl-phenyl)-pyrazolo[1,5-b]pyridazin-2-yl]-phenol

Boron tribromide (1M solution in CH₂Cl₂, 2.1 eq) was added to 3-(4-methanesulfonyl-phenyl)-2-(4-methoxy-phenyl)-pyrazolo[1,5-b]pyridazine (3.58 g) in CH₂Cl₂ at −70°. The mixture was stirred for 10 min then warmed to 0° and stirred at 0° overnight. The reaction mixture was made alkaline with potassium carbonate then acidified with hydrochloric acid (2M), poured into water and extracted into CH₂Cl₂. The organic phase was dried, filtered and concentrated to give the title compound (1.87 g) as a yellow solid.

MH⁺ 366

1H NMR (DMSO) δ 3.30 (3H, s) 6.80 (2H, d, J=8.5 Hz) 7.30 (1H, dd, J=9 & 5 Hz) 7.35 (2H, d, J=8.5 Hz) 7.60 (2H, d, J=8 Hz) 8.0 (2H, d, J=8.5 Hz) 8.20 (1H, dd, J=9 & 2 Hz) 8.55 (1H, dd, J=5 & 2 Hz) 9.75 (1H, s)

(ii) 2-(4-Ethoxy-phenyl)-3-(4-methanesulfonyl-phenyl)-pyrazolol[5-b]pyridazine

4-[3-(4-Methanesulfonyl-phenyl)-pyrazolo[1,5-b]pyridazin-2-yl]-phenol (663 mg, 1.82), iodoethane (1 eq) and potassium carbonate (2 eq) in acetonitrile (30 mL) were heated at reflux under nitrogen for 18 h. The cooled reaction mixture was partitioned between water (30 mL) and ethyl acetate (30 mL). The organic phase was collected, dried and purified by chromatography to give the title compound (547 mg) as a cream foam.

MH⁺ 394

1H NMR (DMSO) δ 1.45 (3H, t, J=7 Hz) 3.10 (3H, s) 4.1 (2H, q, J=7 Hz) 6.87 (2H, d, J=9 Hz) 7.08 (1H, dd, J=9 & 5 Hz) 7.55 (4H, t, J=9 Hz) 7.92 (1H, dd, J=9 & 2 Hz) 7.95 (2H, d, J=9 Hz) 8.20 (1H, dd, J=9 & 2 Hz) 8.32 (1H, dd, J=5 & 2 Hz)

### EXAMPLE 2.4

### 2-(4-Fluoro-phenyl)-6-methanesulfonyl-3-(4-methanesulfonyl-phenyl)-pyrazolo[1,5-]pyridazine

(i) 2-(4-Fluoro-phenyl)-6-methylsulfanyl-pyrazolo[1,5-b]pyridazine-3-carboxylic acid methyl ester

Solid t-butoxycarbonyl-O-mesitylenesulfonylhydroxylamine¹ (7.8 g) was added portionwise with stirring to TFA (25 mL) over 10 min then stirred for a further 20 minutes. The solution was poured onto ice (˜200 mL) and left until the ice melted. The resulting white solid was filtered off, washed with water, and dissolved in DME (100 mL). The solution was dried over 4 A mol. sieves for 1.5 hours, filtered then added to a solution of 3-methylthio-pyridazine² (2.6 g) in dichloromethane (35 mL) and the reaction stirred at room temperature for 20 h. The intermediate salt was isolated by filtration as light brown crystals (3.87 g), suspended in acetonitrile (100 mL) and methyl 3-(4-fluoro-phenyl)-prop-2-ynoic acid (2.02 g) added. 1,8-Diazabicyclo[5.4.0]undec-7-ene (2.1 mL) was added dropwise and the reaction was stirred at room temperature for 20 hours. The resulting crystalline precipitate was filtered off, washed and dried (770 mg). Concentration of the filtrate gave a second crop (430 mg). The residues were partioned between water and ethyl acetate (100 mL each) and the aqueous layer was extracted with ethyl acetate (20 mL). The combined organics were washed with water, brine and dried. Removal of solvent gave a brown oil which was purified by flash chromatography on silica (300 g) eluting with cyclohexane/ethyl acetate (3:1) to give a further quantity of product (247 mg). The three crops were combined to give the title compound (1.45 g) as a light brown solid.

MH⁺ 318

1H NMR (CDCl₃) δ 2.70 (3H, s), 3.88 (3H, s) 7.08-7.18 (3H, m) 7.84 (2H, m) 8.31 (1H, d, J=10 Hz)

Ref:¹ K Novitskii et al, Khim Geterotskil Soedin, 1970 2, 57-62

Ref:² Barlin G. B., Brown, W. V., J Chem Soc (1968), (12),1435-45

(ii) 2-(4-Fluoro-phenyl)-3-(4-methanesulfonyl-phenyl)-6-methylsulfanyl-pyrazolo[1.5-b]pyridazine

A mixture of the 2-(4-fluoro-phenyl)-6-(methylthio)-pyrazolo[1,5-b]pyridazine-3-carboxylic acid methyl ester (1.45 g) potassium carbonate (690 mg) in methanol (40 mL) and water (14 mL) was stirred and heated under reflux for 20 hours under nitrogen. The solvents were removed and the resulting solid partioned between ethyl acetate (50 mL) and water (250 mL). The aqueous layer was acidified to pH1 (2 MHCl) and a solid was filtered off (1.0 g, MH⁺ 304). A mixture of the solid (1.0 g), sodium bicarbonate (557 mg) and NBS (594 mg) were stirred at room temperature for 4 hours. The reaction was poured into water (150 mL) and extracted with ethyl acetate (3×50 mL). The combined extracts were washed with water (50 mL), brine (20 mL), dried and concentrated. The resulting solid (1.015 g, MH⁺ 338,340), 4-(methanesulphonyl)phenyl boronic acid (902 mg), sodium carbonate (740 mg) and tetrakis(triphenylphosphine)palladium(O) (175 mg) were stirred and heated under nitrogen at reflux in DME (30 mLs) and water (15 mL) for 48 hours. The reaction was poured into water and extracted with ethyl acetate (3×50 mL). The combined extracts were dried and the solvent removed to give a brown solid. This was purified on silica (300 g) eluting with cyclohexane, ethyl acetate (1:1) to give the title compound (0.713 g) as a yellow solid.

MH⁺ 414

1H NMR δ (DMSO) 2.65 (3H, s) 3.28 (3H, s) 7.20-7.30 (3H, m) 7.55 (2H, m) 7.62 (4H, d, J=8.5 Hz) 7.95-8.05 (3H, m)

(iii) 2-(4-Fluoro-phenyl)-6-methanesulfonyl-3-(4-methanesulfonyl-phenyl)-pyrazolo[1,5-b]pyridazine

A suspension of 2-(4-fluoro-phenyl)-3-(4-methanesulfonyl-phenyl)-6-(methylthio)-pyrazolo[1,5-b]pyridazine (60 mg 0.145) in MeOH (5 mL) and water (2 mL) was stirred with oxone (196 mg 0.32) for 20 hours. The resulting solution was poured into water (50 mL) and extracted with chloroform (3×20 mL). The combined extracts were dried and the solvent removed. Crystallisation of the residue from methanol gave the title compound (60 mg) as a white solid.

MH⁺ 446

1H NMR (DMSO-d₆) δ 3.34 (3H, s) 3.53 (3H, s) 7.33 (2H, t, J=9 Hz) 7.62 (2H, m) 7.68 (1H, d, J=8.5 Hz) 8.04 (1H, d, J=10 Hz) 8.52 (1H, d, J=9 Hz)

TLC SiO₂ Hexane:Ethyl acetate (1:1) Rf 0.24 UV

### EXAMPLE 2.5

### 2-(4-Difluoromethoxy-phenyl)-3-(4-methanesulfonyl-phenyl)-pyrazolo[1,5-b]pyridazine

Sodium hydride (48 mg, 60% disp. in oil, 1.2 mmol) was added to a solution of 4-[3-(4-methanesulfonyl-phenyl)-pyrazolo[1,5-b]pyridazin-2-yl]-phenol (200 mg, 0.55 mmol) in anhydrous dimethylformamide (5 mL). Bromodifluoromethane gas was gently bubbled through the solution for 20 min, then diluted with CH₂Cl₂ (30 mL). Aqueous workup followed by chromatography on silica gel with CH₂Cl₂:ethyl acetate (3:1) as eluant then chromatography with CH₂Cl₂:ethyl acetate (10:1) as eluant gave the title compound (63 mg, 28%) as a white solid.

MH⁺ 416

NMR (CDCl₃) δ 8.38 (1H, dd, J=4 Hz), 8.01 (2H, d, J=8.5 Hz), 7.94 (1H, dd, J=9 & 2 Hz), 7.65 (2H, d, J=8.5 Hz) 7.57 (2H, d, J=8 Hz), 7.10 (3H, m), 6.87-6.27 (1H, t, J=7.4 Hz) 3.15 (3H, s)

### EXAMPLE 2.6

### 4-[2-(4-Ethoxy-phenyl)-pyrazolol[1,5-b]pyridazin-3-yl]-benzenesulfonamide

(i) 2-(4-Ethoxy-phenyl)-pyrazolo[1,5-b]pyridazine-3-carboxylic acid methyl ester

Diazabicyclo[5.4.0]undec-7-ene (1.47 mL, 2 eq) was added dropwise to a solution of methyl 3-(4-ethoxy-phenyl)-prop-2-ynoic acid (1.0 g) and 1-amino pyridazinium iodide² (2.19 g) in acetonitrile (10 mL) under nitrogen and stirred for 5 h. Concentration and aqueous workup gave the title compound (1.2 g) as a sticky brown solid.

MH⁺ 298

(ii) 2-(4-Ethoxy-phenyl)-pyrazolo[1,5-b]pyridazine-3-carboxylic acid

A mixture of 2-(4-ethoxy-phenyl)-pyrazolo[1,5-b]pyridazine-3-carboxylic acid methyl ester (1.2 g), ethanol (10 mL) and 2N sodium hydroxide (10 mL) was heated to 80° for 1.5 h. The mixture was allowed to cool and acidified to pH 1 with 2N hydrochloric acid. The title compound was isolated by filtration as a brown solid (716 mg, 63%).

MH⁺ 284

(iii) 2-(4-Ethoxy-phenyl)-3-iodo-pyrazolo[1,5-b]pyridazine

A mixture of 2-(4-ethoxy-phenyl)-pyrazolo[1,5-b]pyridazine-3-carboxylic acid (710 mg), N-iodosuccinimide (678 mg) and sodium bicarbonate (717 mg) in DMF (8 mL) was stirred for 4 h. A further quantity of N-iodosuccinimide (100 mg) was added and stirring continued for 2 h. Aqueous workup gave a dark brown solid which was purified by SPE with dichloromethane as eluant. This gave the title compound as an orange-brown solid (429 mg, 47%).

MH⁺ 366

(iv) 4-[2-(4-Ethoxy-phenyl)-pyrazolo[1.5-b]pyridazin-3-yl]-benzenesulfonamide

A mixture of 4-iodobenzenesulphonamide (0.311 g), dipinacoldiborane¹ (0.279 g), potassium acetate (486 mg) and [1,1′-bis(diphenylphosphino)-ferrocene]palladium(II) chloride complex with dichloromethane (1:1) (0.45 g) in dimethylformamide (8 mL) was heated under nitrogen at 80° for 2 h. The cooled reaction mixture was concentrated in vacuo and the residue suspended in 1,2 dimethoxyethane (10 mL), 2-(4-ethoxy-phenyl)-3-iodo-pyrazolo[1,5-b]pyridazine (0.4 g) was added together with 2N sodium carbonate (4 mL) and tetrakis(triphenylphosphine)palladium (O) (20 mg) and the mixture heated at reflux under nitrogen for 18 hours. The cooled reaction mixture was poured into water (60 mL) and the suspension extracted with ethyl acetate (3×60 mL). The organic extracts were combined, dried (Na₂SO₄) and concentrated. The residue was purified by chromatography eluting with dichloromethane/ethyl acetate (3:1) to give the title compound as a yellow solid (0.116 g, 27%).

MH⁺ 395

NMR (CDCl₃) δ 8.32 (1H, dd, J=4 & 2 Hz), 7.97 (2H, d, J=8 Hz), 7.89 (1H, dd, J=9 & 2 Hz), 7.54 (4H, m), 7.04 (1H, dd, J=9 & 4 Hz), 6.88 (2H, d, J=9 Hz), 1.43 (3H, t, J=7 Hz)

Ref:¹ R. Miyaura et al J. Org. Chem., 1995,60,7508-7510.

### EXAMPLE 2.7

### 6-Difluoromethoxy-2-(3-fluoro-phenyl)-3-(4-methanesulfonyl-phenyl)-pyrazolo[1,5-b]pyridazine

(i) 1-(2,2-Dibromo-vinyl)-3-fluoro-benzene

To a stirred cooled (ice/salt, 0°) solution of carbon tetrabromide (48.82 g) in anhydrous CH₂Cl₂ (200 mL) was added portionwise over 3 minutes, triphenylphosphine (77.1 g), maintaining the temperature below 10°. The resulting orange suspension was stirred at 0° for 1 hour before adding to it, 3-fluorobenzaldehyde (7.8 mL). After the addition was complete, the suspension was stirred at 0° for 1 hour then quenched by the addition of water (75 mL). The organic phase was separated and washed with brine (75 mL), dried (Na₂SO₄) and evaporated to dryness. The residual gum was poured into cyclohexane (1 L) and stirred for 30 minutes. The organic phase was decanted and the residue taken up into CH₂Cl₂ and poured into cyclohexane (1 L). This procedure was repeated twice more and the combined organic phases concentrated to ˜100 mL and passed through silica gel. The filtrate was concentrated to give the title compound as a mobile yellow oil (24 g, 100%).

MH⁺ 280, MH⁻ 279

NMR (CDCl₃) δ 7.05 (1H, tm, J=9 Hz) 7.3 (3H, m) 7.45 (1H, s)

(ii) (3-Fluoro-phenyl)-propynoic acid methyl ester

To a stirred solution of 1-(2,2-dibromo-vinyl)-3-fluoro-benzene (23.8 g) in anhydrous THF (350 mL) cooled to −78° was added dropwise over 30 minutes, n-butyllithium (2.2 eq, 1.6 M in hexanes). The mixture was stirred for a further 30 minutes at −78° before methyl chloroformate (11.6 g, 9.5 mL) was added and the resultant mixture allowed to warm to 0° for 1 hour before being diluted with 1:1 saturated aqueous sodium bicarbonate:ammonium chloride (100 mL) and extracted into ether (2×100 mL). The combined organic extract was washed with brine (25 mL), dried (Na₂SO₄) and evaporated to dryness to give the title compound as a brown oil (16.7 g, 100%).

MH⁻ 173

NMR (CDCl₃) δ 7.4-7.1 (4H, m) 3.85 (3H, s, CO₂Me)

(iii) 2-(3-Fluoro-phenyl)-6-methoxy-pyrazolo[1.5-b]pyridazine-3-carboxylic acid methyl ester

1,8-Diazabicyclo[5.4.0]undec-7-ene (5 mL) was added to a stirred, chilled, mixture of (3-fluoro-phenyl)-propynoic acid methyl ester (2.67 g) and 1-amino-3-methoxy-pyridazin-1-ium mesitylene sulphonate (4.89 g) in acetonitrile (80 mL) and the mixture was stirred at 0° for 1 hour then at ambient temperature for 18 hours. The mixture was concentrated in vacuo, and partitioned between ethyl acetate (150 mL) and water (150 mL). The aqueous phase was separated and further extracted with ethyl acetate (2×100 mL). The combined organic extracts were washed with water (2×500 mL), brine (25 mL), dried (MgSO₄), filtered and evaporated in vacuo to give a solid which was triturated with anhydrous ether: petroleum ether (1:0.5) to give the title compound as a brown solid (2.4 g, 53%).

MH⁺ 302

1H NMR (CDCl₃) δ 12.8 (1H, br s); 8.4 (1H, d, J 10 Hz) 7.7-7.6 (2H, m) 7.42 (1H, q, J 8 Hz) 7.15 (1H, td, J 8 & 3 Hz) 6.95 (1H, d, J 10 Hz) 4.1 (3H, s) 3.88 (3H, s)

(iv) 2-(3-Fluoro-phenyl)-6-methoxy-pyrazolo[1,5-b]pyridazine-3-carboxylic acid

2N sodium hydroxide (50 mL) was added to a solution of 2-(3-fluoro-phenyl)-6-methoxy-pyrazolo[1,5-b]pyridazine-3-carboxylic acid methyl ester (2.3 g) in absolute ethanol (50 mL) and the resulting mixture heated to reflux for three hours. The cooled reaction mixture was poured slowly into a stirred solution of 2N hydrochloric acid (300 mL). The resulting suspension was stirred at ambient temperature for 1 hour then filtered and the filter cake washed with water and dried in vacuo at 60° to give the title compound as an off-white solid (2.0 g, 91%).

MH⁺ 288

1H NMR (DMSO) δ 8.45 (1H, d, J 10 Hz); 7.67 (2H, m); 7.5 (1H, q, J 7 Hz); 7.3 (1H, td, J 7 & 2 Hz); 7.21 (1H, d, J 10 Hz); 4.0 (3H, s)

(v) 3-Bromo-2-(3-fluoro-phenyl)-6-methoxy-pyrazolo[1,5-b]pyridazine

To a stirred solution of 2-(3-fluoro-phenyl)-6-methoxy-pyrazolo[1,5-b]pyridazine-3-carboxylic acid (2.0 g) in anhydrous DMF (20 mL) was added n-bromosuccinimide (1.78 g) and the resulting solution stirred at ambient temperature for 3 hours. The reaction mixture was diluted with ethyl acetate (800 mL) and washed sequentially with water (10×100 mL) and sat. brine (25 mL), dried (Na₂SO₄), and concentrated to give the title compound as a buff solid (2.1 g, 93%).

MH⁺ 323, MH⁻ 321

1H NMR (CDCl₃) 7.9 (2H, m) 7.8 (1H, d, J 10 Hz); 7.45 (1H, m); 7.1 91H, td, J 8 & 2 Hz); 6.78 (1H, d, J 10 Hz); 4.1 (3H, s)

(vi) 6-Difluoromethoxy-2-(3-fluoro-phenyl)-pyrazolo[1.5-b]pyridazine

Portions of 3-bromo-2-(3-fluoro-phenyl)-6-methoxy-pyrazolo[1,5-b]pyridazine (400 mg, 2.1 g total) were placed in individual Reactivials equipped with a magnetic stirrer bar. Pyridine hydrochloride (10 eq) was added to each vial, the vials sealed, and heated to 200° for 3 hours. The vials were allowed to cool to ˜140° before opening and the contents poured into ice/water. The resulting mixture was extracted into ethyl acetate (3×100 mL) and the combined organic extracts washed with water (7×75 mL), dried (Na₂SO₄) and evaporated to give the des-bromo phenol as a brown solid (1.0 g, MH⁺ 230). This solid was dissolved in anhydrous DMF (10 mL) and sodium hydride (60% dispersion in mineral oil, 200 mg) added portionwise. After stirring for 20 minutes at ambient temperature the solution was transferred to a small cooled autoclave and bromodifluoromethane (5 mL, xs, condensed at −30°) added. The autoclave was then sealed, allowed to warm to ambient temperature and stirred for 36 hours. The resulting solution was diluted with ethyl acetate (200 mL), washed with water (10×20 mL), dried (Na₂SO₄), concentrated and the residual gum purified by flash column chromatography with cyclohexane:ethyl acetate (4:1) as eluant. This gave the title compound as a solid (652 mg, 60%).

MH⁺ 280 MH⁻ 278

NMR (DMSO) δ 8.42(1H, d, J=10 Hz) 7.85 (1H, d, J 8 Hz) 7.78 (1H, t, J 70 Hz) 7.55 (1H, q, J 8 Hz) 7.38 (1H, s) 7.25 (1H, m) 7.17 (1H, d, J 10 Hz)

(vii) 3-Bromo-6-difluoromethoxy-2-(3-fluoro-phenyl)-pyrazolo[1,5-b]pyridazine

N-bromo succinimide (195 mg) was added to a solution of 6-difluoromethoxy-2-(3-fluoro-phenyl)-pyrazolo[1,5-b]pyridazine (251 mg) and sodium bicarbonate (185 mg) in anhydrous DMF (10 mL) and stirred for 18 h. The reaction mixture was diluted with ethyl acetate (300 mL) and washed with water (10×20 mL), brine (20 mL), dried (Na₂SO₄) and concentrated to give the title compound as a solid (293 mg, 91%).

MH⁺ 359, MH⁻ 356/357

NMR (DMSO) δ 8.36 (1H, d, J 10 Hz) 7.88 (1H, d, J 8 Hz) 7.78 (1H, t, J 70 Hz, OCHF₂) 7.77 (1H, dm, J 10 Hz) 7.62 (1H, dt, J 8 & 6 Hz) 7.38 (1H, dt, J 9 & 2 Hz) 7.3 (1H, d, J 10 Hz)

(viii) 6-Difluoromethoxy-2-(3-fluoro-phenyl)-3-(4-methanesulfonyl-phenyl)-pyrazolo[1,5-b]pyridazine

To a stirred solution of 3-bromo-6-difluoromethoxy-2-(3-fluoro-phenyl)-pyrazolo[1,5-b]pyridazine (286 mg) in DMF (20 mL) was added 2N aq sodium carbonate (10 mL). To this mixture was added 4-methanesulfonyl-phenylboronic acid (180 mg) and tetrakis triphenylphosphine palladium (O) (34 mg). The resulting mixture was stirred and heated to reflux for 18 hours. The cooled reaction mixture was diluted with ethyl acetate (300 mL) and the organic solution washed with water (10×30 mL) and brine (30 mL), dried (Na₂SO₄) and evaporated to give a gum which was purified by flash column chromatography with chloroform:ethyl acetate (50:1 to 5:1) as eluant. Combination of appropriate fractions and concentration gave the title compound as an off-white solid (132 mg, 37%).

MH⁺ 434

1H NMR (CDCl₃) δ 8.02 (1H, d, J 9 Hz); 7.95 (2H, d, J 10 Hz); 7.58 (1H, d, 9 Hz); 7.52 (1H, t, J 70 Hz); 7.32 (3H, m); 7.08 (1H, m); 6.9 (1H, d, J 9 Hz); 3.15 (3H, s)

### EXAMPLE 3

### Preparation of Compounds of Formula (III)

Compounds of formula (III) may be prepared by any method described in WO 2004/024691 and equivalent patent applications.

### EXAMPLE 3.1

### N-cyclohexyl4-(trifluoromethyl)-6-[4-(methylsulfonyl)phenyl]pyridine-2-amine

(i) 2-[4-(methylthio)phenyl]4-(trifluoromethyl)-pyridine

To a mixture of 2-chloro-4-(trifluoromethyl)pyridine (19.9 g, 0.11 mol), 4-(methylthio)phenylboronic acid (21.9 g, 0.13 mol), 1M aqueous sodium carbonate (180 mL) and 1,2-dimethoxyethane (270 mL) under an atmosphere of nitrogen was added palladium tetrakistriphenylphosphine (3.78 g, 3.3 mmol) and the reaction heated at 100° C. for 14 hours. After cooling and concentration in vacuo, the residue was partitioned between ethyl acetate (350 mL) and water (400 mL) and separated. The aqueous layer was further extracted with ethyl acetate (2×150 mL) and the combined organic layers were dried over sodium sulfate and concentrated in vacuo. Filtration through a pad of silica gel (200 g) eluting with a gradient of ethyl acetate in cyclohexane gave the title compound (29.4 g) LC retention time 3.62 mins, MS m/z 269 (MH⁺).

(ii) 2-[4-(methylsulfonyl)phenyl]-4-(trifluoromethyl)-pyridine

To a stirred suspension of intermediate (i) (29.4 g, 0.1 mol) in methanol (400 mL) at 0° C. was added portionwise a suspension of Oxone™ (134 g) in water (200 mL). The reaction was warmed to room temperature and stirred for 14 hours. The methanol was removed in vacuo and the residue diluted with saturated aqueous sodium bicarbonate (2 L) and extracted with ethyl acetate (3×1 L). The combined organic layers were dried over sodium sulfate and concentrated in vacuo to give the title compound (32 g, 0.106 mol) LC retention time 2.90, MS m/z 302 (MH⁺)

(iii) 2-Chloro-4-(trifluoromethyl)-6-[4-(methylsulfonyl)phenyl] pyridine

To a solution of intermediate (ii) (32 g, 0.106 mol) in dichloromethane (400 mL) at reflux was added 3-chloroperbenzoic acid (41.7 g of 57 to 86% grade material) portionwise over 15 minutes. After stirring for 14 hours at reflux, the reaction was cooled, diluted with dichloromethane (2 L) and washed sequentially with saturated aqueous sodium bicarbonate solution, saturated aqueous sodium sulfite solution containing tetra-n-butylammonium sulfate (4 mL) and water, dried over sodium sulfate and concentrated in vacuo to give 2-[4-(methylsulfonyl)phenyl]-4-(trifluoromethyl)-pyridine-N-oxide (37.2 g, containing traces of a tetra-n-butylammonium salt) LC retention time 2.34, MS m/z 318 (MH⁺). A mixture of this crude material and phosphorus oxychloride (110 mL) was heated at 110° C. for 4 hours. After cooling, the majority of the phosphorus oxychloride was removed in vacuo and the residue neutralised with saturated aqueous sodium bicarbonate solution (300 mL), with cooling. The mixture was extracted with chloroform and the combined organic extracts dried over sodium sulfate and concentrated in vacuo. The residue was recrystallised from 2-propanol to give the title compound (22.0 g) LC retention time 3.23 min, MS m/z 336/338 (MH⁺).

(iv) N-cyclohexyl-4-(trifluoromethyl)-6-[4-(methylsulfonyl)phenyl]pyridine-2-amine

A stirred mixture of intermediate (iii) (6 g, 17.8 mmol) and cyclohexylamine (175 mL) was heated at 110° C. for 14 hours. After cooling, the reaction was diluted with water (1 L), acidified with 2N HCl (750 mL) and filtered to give the title compound (6.48 g) LC retention time 3.81 mins MS m/z 399 (MH⁺); ¹H-NMR (CDCl₃) δ 1.22-1.86 (8H, m), 2.60-2.16 (2H, m), 3.09 (3H, s), 3.67-3.78 (1H, m), 4.84 (1H, d, J=7 Hz), 6.57 (1H, s), 7.19 (1H, s), 8.03 (2H, d, J=9 Hz), 8.17 (2H, d, J=9 Hz).

### EXAMPLE 3.2

### 2-[4-(methylsulfonyl)phenyl]-6-[(2-pyridinylmethyl)oxy]-4-(trifluoromethyl)pridine

(i) 4-(Trifluoromethyl)-6-[4-(methylthio)phenyl]-2-pyridone

To a stirred solution of diisopropylamine (11.5 mL, 81.8 mmol) in THF (75 mL) at 0° C. was added n-butyllithium (51.1 mL of a 1.6M solution in hexanes, 81.8 mmol). After stirring for 15 minutes, a solution of 4,4,4-trifluoro-3-methyl-2-butenoic acid (6.0 g, 38.9 mmol) in THF (10 mL) was added dropwise. The reaction was allowed to warm to room temperature and stirred for 30 minutes before being cooled to 0° C. and treated dropwise with a solution of 4-(methylthio)benzonitrile (2.91 g, 19.5 mmol) in THF (10 mL). Upon complete addition, the reaction was heated at reflux for 14 hours. After cooling, water (200 mL) was added and the mixture extracted with ethyl acetate (250 mL). The organic phase was dried over sodium sulfate, filtered and concentrated in vacuo and the resulting residue purified by silica chromatography eluting with 1:1 ethyl acetate / cyclohexane to give the title product (2.43 g) LC retention time 3.10 mins MS m/z 286 (MH⁺).

(ii) 4-(Trifluoromethyl)-6-[4-(methylsulfonyl)phenyl]-2-pyridone

To a stirred mixture of intermediate (i) (2.43 g, 8.52 mmol) in methanol (100 mL) at 0° C. was added portionwise a suspension of Oxone™ (15.7 g, 25.6 mmol) in water (60 mL). The reaction was warmed to room temperature and stirred for 14 hours. The methanol was removed in vacuo and the resulting residue partitioned between saturated aqueous sodium bicarbonate (500 mL) and chloroform (200 mL) and separated. The aqueous layer was further extracted with chloroform (3×100 mL) and the combined organic layers were dried over sodium sulfate, filtered and concentrated to give the title compound (1.72 g) LC retention time 2.57 mins, MS m/z 318 (MH⁺).

(iii) 2-[4-(methylsulfonyl)phenyl]-6-[(2-pyridinylmethyl)oxy]-4-(trifluoromethyl)pyridine

Diisopropylazodicarboxylate (0.93 mL, 4.7 mmol) was added dropwise to a solution of intermediate (ii) (1 g, 3.2 mmol), 2-pyridinylmethanol (0.38 mL, 3.9 mmol) and triphenylphosphine (1.24 g, 4.7 mmol) in chloroform (80 mL). After stirring for 14 hours, the reaction was concentrated and the residue diluted with methanol and loaded onto a methanol-conditioned 10 g Varian bond-elut SCX-2 cartridge. The cartridge was washed with methanol (2×40 mL) followed by a solution of 9:1 methanol/2N hydrochloric acid. The combined acidic fractions were concentrated and the residue triturated with methanol to give the title compound as its hydrochloride salt (348 mg) LC retention time 3.35 mins, MS m/z 409 (MH⁺); ¹H-NMR (d₆-DMSO) δ 3.28 (3H, s), 5.79 (2H, s), 7.47 (1H, s), 7.64 (1H, t, J=6 Hz), 7.85 (1H, d, J=8 Hz), 8.03 (2H, d, J=9 Hz), 8.11 (1H, s), 8.17 (1H, t, J=8 Hz), 8.38 (2H, d, J=9 Hz), 8.75 (1H, d, J=6 Hz)

### EXAMPLE 3.3

### 4-methyl-N-[(1-methyl-1H-pyrazol-4-yl)methyl]-6-[4-(methylsulfonyl)phenyl]-2-pyridinamine

(i) 4-Methyl-6-[4-(methylthio)phenyl]-2-pyridone

To a stirred solution of lithium diisopropylamide (50 mL of a 2M solution in heptane/THF/ethyl benzene, 0.1 mol) in THF (50 mL) at −78° C. and under an atmosphere of nitrogen was added dropwise a solution of 3-methyl-2-butenoic acid (5 g, 0.05mol) in THF (50 mL). The reaction was warmed to 0° C. for 30 minutes. After cooling to −78° C., a solution of 4-(methylthio)benzonitrile (7.45 g, 0.05 mol) in THF (50 mL) was added dropwise. Upon complete addition, the reaction was warmed to room temperature and stirred for 3 hours. Water (150 mL) and ethyl acetate (100 mL) were added to the reaction mixture and the resulting precipitate filtered, washed with ethyl acetate and dried to give the title compound (4.96 g, 43%) LC retention time 2.75 mins, MS m/z 232 (MH⁺).

(ii) 4-Methyl-6-[4-(methylsulfonyl)phenyl]-2-pyridone

To a stirred mixture of intermediate (i) (3.7 g, 16.0 mmol) in methanol (150 mL) at 0° C. was added portionwise a suspension of Oxone™ (29.5 g, 48.0 mmol) in water (100 mL). The reaction was warmed to room temperature and stirred for 14 hours. The methanol was removed in vacuo and the resulting residue partitioned between saturated aqueous sodium bicarbonate (1 L) and chloroform (500 mL) and separated. The aqueous layer was further extracted with chloroform (3×200 mL) and the combined organic layers were dried over sodium sulfate, filtered and concentrated to give the title compound (3.20 g, 76%) LC retention time 2.20 mins, MS m/z 264 (MH⁺).

(iii) 4-Methyl-6-[4-(methylsulfonyl)phenyl]pyridine-2-trifluoromethanesulfonate

To a stirred solution of intermediate (ii) (3.20 g, 12.2 mmol) in pyridine (150 mL) at 0° C. and under an atmosphere of nitrogen was added dropwise trifluoromethanesulfonic anhydride (2.46 mL, 14.6 mmol). After stirring for 1 hr at 0° C., the pyridine was removed in vacuo and the residue partitioned between water (200 mL) and dichloromethane (200 mL). The layers were separated and the aqueous phase further extracted with dichloromethane (3×100 mL). The combined organic layers were dried over sodium sulfate, filtered and concentrated in vacuo to give the title compound (4.27 g, 89%) LC retention time 3.48 mins, MS m/z 396 (MH⁺).

(iv) N-[(1-methyl-1H-pyrazol-4-yl)methyl]-4-methyl-6-[4-(methylsulfonyl)phenyl]pyridine-2-amine

A stirred solution of intermediate (iii) (1.25 g, 3.15 mmol) and (1-methyl-1H-pyrazol-4-yl)methylamine (0.70 g, 6.30 mmol) in NMP (10 mL) was heated at 180° C. for 14 hours, cooled, and loaded evenly onto 5 methanol-conditioned 10 g Varian bond-elut SCX-2 cartridge. The cartridges were washed with methanol (2×400 mL each) followed by a solution of 9:1 methanol/concentrated ammonium hydroxide (2×40 mL each). The ammoniacal fractions were concentrated and purified by silica chromatography eluting with a gradient of cyclohexane to ethyl acetate to give the title compound (780 mg) LC retention time 2.32 mins, MS m/z 357 (MH⁺); ¹H-NMR (CDCl₃) δ 2.23 (3H, s), 3.09 (3H, s), 3.88 (3H, s), 4.47 (2H, d, J=6 Hz), 4.68 (1H, br), 6.28 (1H, s), 6.99 (1H, s), 7.36 (1H, s), 7.50 (1H, s), 8.00 (2H, d, J=9 Hz), 8.19 (2H, d, J=9 Hz).

### EXAMPLE 4

### Biological Data

Inhibitory activity against human COX-1 and COX-2 was assessed in COS cells which had been stably transfected with cDNA for human COX-1 and human COX-2. 24 Hours prior to experiment, COS cells were transferred from the 175 cm² flasks in which they were grown, onto 24-well cell culture plates using the following procedure. The incubation medium

(Dulbecco's modified eagles medium (DMEM) supplemented with heat-inactivated foetal calf serum (10% v/v), penicillin (100 lU/ml), streptomycin (100 μg/ml) and geneticin (600 μg/ml)) was removed from a flask of confluent cells (1 flask at confluency contains approximately 1×10⁷ cells). 10 ml of phosphate buffered saline (PBS) was added to the flask to wash the cells. Having discarded the PBS, cells were then rinsed in 10 ml trypsin for 20 seconds, after which the trypsin was removed and the flask placed in an incubator (37°) for 1-2 minutes until cells became detached from the flask. The flask was then removed from the incubator and cells resuspended in 10 ml of fresh incubation medium. The contents of the flask was transferred to a 250 ml sterile container and the volume of incubation medium subsequently made up to 100 ml. 1 ml cell suspension was pipetted into each well of 4×24-well cell culture plates. The plates were then placed in an incubator (37° C., 95% air/5% CO₂) overnight. If more than 1 flask of cells were required, the cells from the individual flasks were combined before being dispensed into the 24-well plates.

Following the overnight incubation, the incubation medium was completely removed from the 24-well cell culture plates and replaced with 250 μl fresh DMEM (37° C.). The test compounds were made up to 250× the required test concentration in DMSO and were added to the wells in a volume of 1 μl. Plates were then mixed gently by swirling and then placed in an incubator for 1 hour (37° C., 95% air/5% CO₂). Following the incubation period, 10 μl of arachidonic acid (750 μM) was added to each well to give a final arachidonic acid concentration of 30 μM. Plates were then incubated for a further 15 minutes, after which the incubation medium was removed from each well of the plates and stored at −20° C., prior to determination of prostaglandin E₂ (PGE2) levels using enzyme immunoassay. The inhibitory potency of the test compound was expressed as an IC₅₀ value, which is defined as the concentration of the compound required to inhibit the PGE2 release from the cells by 50%. The selectivity ratio of inhibition of COX-1 versus COX-2 was calculated by comparing respective IC₅₀ values.

The following IC₅₀ values for inhibition of COX-2 and COX-1 were obtained for compounds of the invention:

| Compound No. | COX-2: IC₅₀(nM) | COX-1: IC₅₀(nM) |
|---|---|---|
| 1.1 | <1 | 81,300 |
| 1.2 | 23 | 9,675 |
| 1.3 | 4 | 2,923 |
| 1.5 | 6 | 61,380 |
| 2.1(v) | 35 | >100,000 |
| 2.2(ii) | <10 | 3,880 |
| 2.3(ii) | 3 | >100,000 |
| 2.4(iii) | 370 | >100,000 |
| 2.5 | 21 | >100,000 |
| 2.6(iv) | 0.44 | 3828 |
| 2.7(viii) | 16 | >55,200 |

### EXAMPLE 5

### Microsomal Assay

Inhibitory activity against microsomal h-COX2 was assessed against a microsomal preparation from baculovirus infected SF9 cells. An aliquot of microsomal preparation was thawed slowly on ice and a 1/40,000 dilution prepared from it into the assay buffer (sterile water, degassed with argon containing 100 mM HEPES (pH 7.4), 10 mM EDTA (pH7.4), 1 mM phenol, 1 mM reduced glutathione, 20 mg/ml gelatin and 0.001 mM Hematin). Once diluted the enzyme solution was then sonicated for 5 seconds (Branson sonicator, setting 4, 1 cm tip) to ensure a homogeneous suspension. 155 μl enzyme solution was then added to each well of a 96-well microtitre plate containing either 5 μl test compound (40× required test concentration) or 5 μl DMSO for controls. Plates were then mixed and incubated at room temperature for 1 hour. Following the incubation period, 40 μl of 0.5 μM arachidonic acid was added to each well to give a final concentration of 0.1 μM. Plates were then mixed and incubated for exactly 10 minutes (room temperature) prior to addition of 25 μl 1M HCl (hydrochloric acid) to each well to stop the reaction. 25 μl of 1M NaOH (sodium hydroxide) was then added to each well to neutralise the solution prior to determination of PGE₂ levels by enzyme immunoassay (EIA).

The following IC₅₀ values for inhibition of COX-2 and COX-1 were obtained from the microsomal assay for compounds of the invention:

| Example No. | COX-2: IC₅₀(nM) | COX-1: IC₅₀(nM) |
|---|---|---|
| 1.6 | <10 | 3,752 |
| 1.7 | <10 | 79,889 |
| 1.8 | <10 | 1,860 |
| 1.9 | 22 | 69,000 |
| 1.10 | 22 | >30000 |

Examples 3.1, 3.2, 3.3 had IC₅₀ values for inhibition of COX-2 of 0.5 μM or less and at least a 100-fold selectivity for COX-2 over COX-1, based on comparison of the respective IC₅₀ values.

### EXAMPLE 6

### Depression/Anxiety Study

Activity of the compounds (I), (II) or (III), in combination with SSRI inhibitors or alternative compounds, vs. depression/anxiety may be evaluated according to the following models:
Porsolt test in mouse for SSRI/TCA (tricyclic antidepressants) (Porsolt et al 1977, Arch Int Pharmacodyn Ther,: 229, 327-336);
Chronic mild stress in rat for SSRI/TCA (Willner, 1991, TiPS,: 12, 131-136);
Maternal deprivation in rat pups for SSRI (or modulator of serotonin receptors)/TCA (Gardner, 1985, J. Pharmacol. Methods 14: 181-187);
Rat social interaction after chronic treatment with SSRI/TCA (File, 1980 J. Neurosci Methods, 2:219-238; Lightowler et al., 1994, Pharmacol., Biochem. Behaviour,: 49, 281-285);
Gerbil social interaction after chronic treatment with SSRI (or modulator of serotonin receptors)/TCA (File, 1997, Pharmacol. Biochem. Behav. 58: 747-752);
Chronic Inescapable Shock in Rats: (Gambarana, C., Ghiglieri, O., Taddei, I., Tagliamonte, A. & De Montis, M. G. (1995). Imipramine and fluoxetine prevent the stress-induced escape deficits in rats through a distinct mechanism of action. Behavioural Pharmacol., 6, 66-73);
Human Marmoset Threat Test: (Barros, M. & Tomaz, C. (2002). Non-human primate models for investigating fear ands anxiety. Neurosci. Biobehav. Rev., 26(2), 187-201).

The Chronic Inescapable Shock in Rats model, developed from the learned Helplessness paradigm, was used to investigate the acquisition of shock-induced escape deficits in rats in the absence and presence of SSRI±COX-2 inhibitors. The test was performed over a period of seven days as an adaptation of the methodology used previously by Gambarana, C., Ghiglieri, O., Taddei, I., Tagliamonte, A. & De Montis, M. G. (1995).

Experiments were carried out on male Sprague-Dawley rats (Charles River, Como, Italy). Animals were kept in a controlled environment with a constant temperature of 22° C. and a 12 hour light/12 hour inverted dark cycle, with free access to food and water. The procedures used in this study for all animals were in strict accordance with the European legislation on the use and care of laboratory animals (CEE N° 86/609) and experiments were performed under red light.

The experimental procedure consists of a pre-test session (exposure to an unavoidable stress of minimum intensity and duration required to induce a reliable behavioural modification) followed 24 hours later by an escape test (for the assessment of the induced behavioural modification).

During the pre-test each rat, immobilised by a flexible wire net, receives 80 electric shocks (1 mA35 s, one every 30 sec) in about 50 min (40 min for the delivery of 80 electric shocks plus 6 min and 40 sec corresponding to the 80×5 sec duration of each shock) through an electrode connected to an S48 Grass Stimulator and applied to the distal third of the tail. The electrode is fixed to the rat's tail with adhesive tape. Twenty four hours later, rats are tested in a shock-escape paradigm in a Plexiglas cage (30×60×30 cm) with dark walls and a floor fitted with stainless steel rods. An electrode is applied to the tail, fixed with adhesive tape, and the electrode tail is covered by flexible plastic tubing. The animal is then placed in the Plexiglas cage which is divided into two equal chambers (by a dark Plexiglas partition with a 10310 cm sliding door), one disconnected from the tail electrode (neutral chamber) and the other connected with it (electrified chamber). After a 5 min habituation period, the animal in the electrified chamber receives 30 consecutive electric shocks (1 mA35 s), at 30 sec intervals. During the delivery of each shock the door connecting the electrified chamber to the neutral one is open. The intensity of the electric shock is graduated in a way that it is almost dispersed through the grid floor and, thus, selectively perceived on the rat's tail. Animals that are perceived to escape, upon exposure to each electric shock, move into the neutral chamber.

Animals spend 30 min a day for at least 3 days in the experimental cage with the sliding door open to become familiar with the test environment, during the week preceding the pre-test. Control rats, never exposed to stress (naive) and made familiar with the test apparatus, have the electrode applied to the tail only during the escape test. At the escape test they typically make an average of 26 escapes out of 30 consecutive trials. A group of rats is also exposed to the sequence of pre-test and escape test. To ensure that a stress-induced escape deficit is typically present in 90% of animals 24 hours after the pre-test, animals scoring 0-8 escapes out of 30 trials were selected. These animals were divided into three additional subgroups and exposed to either vehicle (0.5% methocel) alone, paroxetine alone (5 mg/kg) or the combination of paroxetine (5 mg/kg p.o.) and 2-butoxy-4-[4-(methylsulfonyl)phenyl]-6-(trifluoromethyl)pyrimidine (10 mg/kg p.o.) for the duration of 7 days of treatment and tested for escape deficit on day 8. Escape deficit is maintained in each animal for chronic stress procedures by 10 min of restraint stress 48 hours after the last escape attempt, receiving 10 min of restraint stress and 4 unavoidable shocks an additional 48 hours later, spending 20 minutes in the cage after an additional 48 hours and repeating on alternate days.

The following Table reports the results obtained testing the combination of paroxetine (5 mg/kg p.o.) and 2-butoxy-4-[4-(methylsulfonyl)phenyl]-6-(trifluoromethyl)pyrimidine (10 mg/kg p.o.) after 7 days of treatment in comparison to paroxetine (5 mg/kg p.o.) alone.

| | MEAN | |
|---|---|---|
| | Number of | |
| | escapes | N |
| NAIVE | 22.9 | 9 |
| STRESS | 2.6 | 6 |
| PAROXETINE | 3.8 | 10 |
| COMBINATION | 22.5 | 7 |

Control rats, never exposed to stress (NAIVE) and made familiar with the test apparatus, make an average of 22.9 escapes out of 30 consecutive trials on day 8. Rats exposed to stress (STRESS), and made familiar with the test apparatus; make an average of 2.6 escapes out of 30 consecutive trials on day 8. Rats exposed to stress and an SSRI (PAROXETINE) after 7 days of treatment, and made familiar with the test apparatus; make 3.8 escapes out of 30 consecutive trials on day 8. This is a similar number of escape attempts to that of the stress group, confirming a lack of reversal of chronic inescapable shock by paroxetine alone. Rats exposed to paroxetine and 2-butoxy-4-[4-(methylsulfonyl)phenyl]-6-(trifluoromethyl)pyrimidine (COMBINATION) after 7 days of treatment, and made familiar with the test apparatus; make 22.5 escapes out of 30 consecutive trials on day 8. The combination therefore highlights a full reversal of the chronic escape deficit. The combination of an antidepressant and COX-2 inhibitor therefore has the potential to have an increased speed of onset to reverse this inescapable shock compared to an antidepressant alone.

All publications, including but not limited to patents and patent applications, cited in this specification are herein incorporated by reference as if each individual publication were specifically and individually indicated to be incorporated by reference herein as though fully set forth.

It is to be understood that the present invention covers all combinations of particular and preferred groups described herein above.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

What is claimed is:

1. A method for the treatment of a depressive disorder in a mammal in need thereof, said method comprising administering to said patient an effective amount of a compound of formula (I) 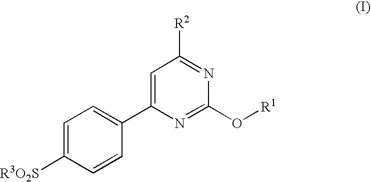
or a pharmaceutically acceptable salt or solvate thereof, in which:
R¹ is selected from the group consisting of H, C₁₋₆alkyl, C₁₋₂alkyl substituted by one to five fluorine atoms, C₃₋₆alkenyl, C₃₋₆alkynyl, C₃₋₁₀cycloalkylC₀₋₆alkyl, C₄₋₁₂bridged cycloalkyl, A(CR⁴R⁵)ₙ and B(CR⁴R⁵)ₙ;
R² is C₁₋₂alkyl substituted by one to five fluorine atoms;
R³ is selected from the group consisting of C₁₋₆alkyl, NH₂ and R⁷CONH;
R⁴ and R⁵ are independently selected from H or C₁₋₆alkyl;
A is selected from the group consisting of unsubstituted 5- or 6-membered heteroaryl, unsubstituted 6-membered aryl, 5- or 6-membered heteroaryl substituted by one or more R⁶ and 6-membered aryl substituted by one or more R⁶;
R⁶ is selected from the group consisting of halogen, C₁₋₆alkyl, C₁₋₆alkyl substituted by one more fluorine atoms, C₁₋₆alkoxy, C₁₋₆alkoxy substituted by one or more F, NH₂SO₂ and C₁₋₆alkylSO₂;
B is a ring selected from the group consisting of 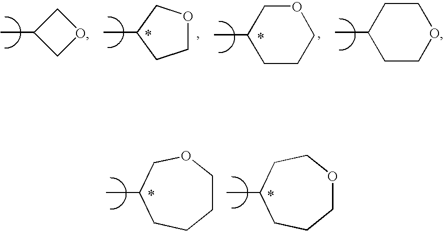
where [symbol] defines the point of attachment of the ring;
R⁷ is selected from the group consisting of H, C₁₋₆alkyl, C₁₋₆alkoxy, C₁₋₆alkylOC₁₋₆alkyl, phenyl, HO₂CC₁₋₆alkyl, C₁₋₆alkylOCOC₁₋₆alkyl, C₁₋₆alkylOCO, H₂NC₁₋₆alkyl, C₁₋₆alkylOCONHC₁₋₆alkyl and C₁₋₆alkylCONHC₁₋₆alkyl; and
n is 0 to 4.

2. A method for the treatment of a depressive disorder in a mammal in need thereof, said method comprising administering to said patient an effective amount of a compound of formula (II) 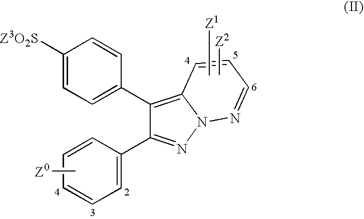
or a pharmaceutically acceptable salt or solvate thereof in which:
Z⁰ is selected from the group consisting of halogen, C₁₋₆alkyl, C₁₋₆alkoxy, C₁₋₆alkoxy substituted by one or more fluorine atoms, and O(CH₂)ₙNZ⁴Z⁵;
Z¹ and Z² are each the same or different and are independently selected from the group consisting of H, C₁₋₆alkyl, C₁₋₆alkyl substituted by one or more fluorine atoms, C₁₋₆alkoxy, C₁₋₆hydroxyalkyl, SC₁₋₆alkyl, C(O)H, C(O)C₁₋₆alkyl, C₁₋₆alkylsulphonyl, C₁₋₆alkoxy substituted by one or more fluorine atoms, O(CH₂)ₙCO₂C₁₋₆alkyl, O(CH₂)ₙSC₁₋₆alkyl, (CH₂)ₙNZ⁴Z⁵, (CH₂)ₙSC₁₋₆alkyl and C(O)NZ⁴Z⁵; with the proviso that when Z⁰ is at the 4-position and is halogen, then at least one of Z¹ and Z² is C₁₋₆alkylsulphonyl, C₁₋₆alkoxy substituted by one or more fluorine atoms, O(CH₂)ₙCO₂C₁₋₆alkyl, O(CH₂)ₙSC₁₋₆alkyl, (CH₂)ₙNZ⁴Z⁵, (CH₂)ₙSC₁₋₆alkyl or C(O)NZ⁴Z⁵;
Z³ is C₁₋₆alkyl or NH₂;
Z⁴ and Z⁵ are each the same or different and are independently selected from the group consisting of H, or C₁₋₆alkyl or, Z⁴ and Z⁵ together with the nitrogen atom to which they are bound, form a 4-8 membered saturated heterocyclic ring having 1 or 2 heteroatoms selected from N, O and S; and
n is 1-4.

3. A method for the treatment of a depressive disorder in a mammal in need thereof, said method comprising administering to said patient an effective amount of a compound of formula (III) 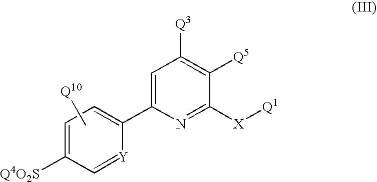
or a pharmaceutically acceptable salt thereof in which:
X is selected from the group consisting of oxygen or NQ²;
Y is selected from the group consisting of CH or nitrogen;
Q¹ is selected from the group consisting of H, C₁₋₆alkyl, C₁₋₂alkyl substituted by one to five fluorine atoms, C₁₋₃alkylOC₁₋₃alkyl, C₃₋₆alkenyl, C₃₋₆alkynyl, C₃₋₁₀cycloalkylC₀₋₆alkyl, C₄₋₇cycloalkyl substituted by C₁₋₃alkyl or C₁₋₃alkoxy, C₄₋₁₂bridged cycloalkyl, A(CR⁶R⁷)ₙ and B(CR⁶R⁷)ₙ;
Q² is selected from the group consisting of H and C₁₋₆alkyl; or
Q¹ and Q² together with the nitrogen atom to which they are bound form a 4-8 membered saturated heterocyclic ring or a 5-membered heteroaryl ring heteroaryl ring is unsubstituted or substituted by one R⁸;
Q³ is selected from the group consisting of C₁₋₅alkyl and C₁₋₂alkyl substituted by one to five fluorine atoms;
Q⁴ is selected from the group consisting of C₁₋₆alkyl, NH₂ and R⁹CONH;
Q⁵ is selected from the group consisting of hydrogen, C₁₋₃alkyl, C₁₋₂alkyl substituted by one to five fluorine atoms, C₁₋₃alkylO₂C, halogen, cyano, (C₁₋₃alkyl)₂NCO, C₁₋₃alkylS and C₁₋₃alkylO₂S;
Q⁶ and Q⁷ are independently H or C₁₋₆alkyl;
A is selected from the group consisting of unsubstituted 5- or 6-membered heteroaryl unsubstituted 6-membered aryl, 5- or
6-membered heteroaryl substituted by one or more R⁸; and 6-membered aryl substituted by one or more R⁸;
Q⁸ is selected from the group consisting of halogen, C₁₋₆alkyl, C₁₋₆alkyl substituted by one more fluorine atoms, C₁₋₆alkoxy, C₁₋₆alkoxy substituted by one or more F, NH₂SO₂ and C₁₋₆alkylSO₂;
B is a ring selected from the group consisting of 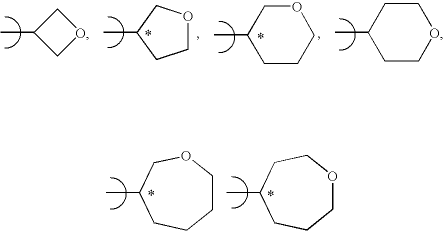
and where [symbol] defines the point of attachment of the ring;
Q⁹ is selected from the group consisting of H, C₁₋₆alkyl, C₁₋₆alkoxy, C₁₋₆alkylOC₁₋₆alkyl, phenyl, HO₂CC₁₋₆alkyl, C₁₋₆alkylOCOC₁₋₆alkyl, C₁₋₆alkylOCO, H₂NC₁₋₆alkyl, C₁₋₆alkylOCONHC₁₋₆alkyl and
C₁₋₆alkylCONHC₁₋₆alkyl;
Q¹⁰ is selected from the group consisting of H and halogen; and
n is 0 to 4.

4. The method of claim 1, further comprising combination with a selective serotonin reuptake inhibitor.

5. A method for the treatment of a depressive disorder in a mammal in need thereof, said method comprising administering to said patient an effective amount of a compound selected from the group consisting of:
2-(4-fluorophenoxy)-4-[4-(methylsulfonyl)phenyl]-6-(trifluoromethyl)pyrimidine;
2-(4-methoxyphenoxy)-4-[4-(methylsulfonyl)phenyl]-6-trifluoromethyl)pyrimidine;
2-butoxy-4-[4-(methylsulfonyl)phenyl]-6-(trifluoromethyl)pyrimidine;
2-[(5-chloropyridin-3-yl)oxy]-4-[4-(methylsulfony)phenyl]-6-(trifluoromethyl)pyrimidine;
2-(cyclohexyloxy)-4-[4-(methylsulfonyl)phenyl]-6-(trifluoromethyl)pyrimidine;
3-(4-methylsulfonyl-phenyl)-2-(4-methoxy-phenyl)-pyrazolo[1,5-b]pyridazine;
6-difluoromethoxy-2-(4-fluoro-phenyl)-3-(4-methylsulfonyl-phenyl)-pyrazolo[1,5-b]-pyridazine;
2-(4-ethoxy-phenyl)-3-(4-methylsulfonyl-phenyl)-pyrazolo[1,5-b]pyridazine;
2-(4-fluoro-phenyl)-6-methylsulfonyl-3-(4-methylsulfonyl-phenyl)-pyrazolo[1,5-b]pyridazine;
2-(4-difluoromethoxy-phenyl)-3-(4-methylsulfonyl-phenyl)-pyrazolo[1,5-b]pyridazine;
4-[2-(4-ethoxy-phenyl)-pyrazolo[1,5-b]pyridazin-3-yl]-benzenesulfonamide;
6-difluoromethoxy-2-(3-fluoro-phenyl)-3-(4-methylsulfonyl-phenyl)-pyrazolo[1,5-b]pyridazine;
3-(4-methanesulfonyl-phenyl)-2-(4-methoxy-phenyl)-pyrazolo[1,5-b]pyridazine;
6-difluoromethoxy-2-(4-fluoro-phenyl)-3-(4-methanesulfonyl-phenyl)-pyrazolo[1,5-b]pyridazine;
2-(4-ethoxy-phenyl)-3-(4-methanesulfonyl-phenyl)-pyrazolo[1,5-b]pyridazine;
2-(4-fluoro-phenyl)-6-methanesulfonyl-3-(4-methanesulfonyl-phenyl)-pyrazolo[1,5-b]pyridazine;
2-(4-difluoromethoxy-phenyl)-3-(4-methanesulfonyl-phenyl)-pyrazolo[1,5-b]pyridazine;
4-[2-(4-ethoxy-phenyl)-pyrazolo[1,5-b]pyridazin-3-yl]-benzenesulfonamide;
6-difluoromethoxy-2-(3-fluoro-phenyl)-3-(4-methanesulfonyl-phenyl)-pyrazolo[1,5-b]pyridazine
4-ethyl-6-[4-(methylsulfonyl)phenyl]-N-(tetrahydro-2H-pyran-4-yl methyl)-2-pyridinamine;
4-methyl-N-[(1-methyl-1H-pyrazol-4-yl)methyl]-6-[4-(methylsulfonyl)phenyl]-2-pyridinamine;
N-[(1,5-dimethyl-1H-pyrazol-4-yl)methyl]-4-methyl-6-[4-(methylsulfonyl)phenyl]-2-pyridinamine;
N-[(1,3-dimethyl-1H-pyrazol-4-yl)methyl]-4-methyl-6-[4-(methylsulfonyl)phenyl]-2-pyridinamine;
4-(6-{[(1,3-dimethyl-1H-pyrazol-4-yl)methyl]amino}-4-ethyl-2-pyridinyl)benzenesulfonamide;
N-[(1,3-dimethyl-1H-pyrazol-4-yl)methyl]-6-[4-(methylsulfonyl)phenyl]-4-(trifluoromethyl)-2-pyridinamine;
N-[(1,5-dimethyl-1H-pyrazol-4-yl)methyl]-6-[4-(methylsulfonyl)phenyl]-4-(trifluoromethyl)-2-pyridinamine;
4-{4-methyl-6-[(tetrahydro-2H-pyran-4-ylmethyl)amino]-2-pyridinyl}-benzenesulfonamide;
4-methyl-N-[(1-methyl-1H-pyrazol-3-yl)methyl]-6-[4-(methylsulfonyl)phenyl]-2-pyridinamine;
N-(cyclohexylmethyl)-6-[4-(methylsulfonyl)phenyl]-4-(trifluoromethyl)-2-pyridinamine;
N-cyclohexyl-6-[4-(methylsulfonyl)phenyl]-4-(trifluoromethyl)-2-pyridinamine;
2-[4-(methylsulfonyl)phenyl]-6-[(2-pyridinylmethyl)oxy]-4-(trifluoromethyl)pyridine;
4-methyl-N-[(3-methyl-4-isoxazolyl)methyl]-6-[4-(methylsulfonyl)phenyl]-2-pyridinamine;
6-[4-(methylsulfonyl)phenyl]-N-(2-pyridinylmethyl)-4-(trifluoromethyl)-2-pyridinamine;
N-cycloheptyl-6-[4-(methylsulfonyl)phenyl]-4-(trifluoromethyl)-2-pyridinamine;
N-(cis-4-methylcyclohexyl)-6-[4-(methylsulfonyl)phenyl]-4-(trifluoromethyl)-2-pyridinamine;
N-(1-ethylpropyl)-6-[4-(methylsulfonyl)phenyl]-4-(trifluoromethyl)-2-pyridinamine;
N-[(3-methyl-1,2,4-oxadiazol-5-yl)methyl]-6-[4-(methylsulfonyl)phenyl]-4-(trifluoromethyl)-2-pyridinamine;
N-[(5-methyl-1,2,4-oxadiazol-3-yl)methyl]-6-[4-(methylsulfonyl)phenyl]-4-(trifluoromethyl)-2-pyridinamine;
4-methyl-N-[(1-methyl-1H-pyrazol-5-yl)methyl]-6-[4-(methylsulfonyl)phenyl]-2-pyridinamine;
N-(cyclopentylmethyl)-6-[4-(methylsulfonyl)phenyl]-4-(trifluoromethyl)-2-pyridinamine;
N-[(1-ethyl-1H-1,2,4-triazol-5-yl)methyl]-4-methyl-6-[4-(methylsulfonyl)phenyl]-2-pyridinamine;
4-ethyl-6-[4-(methylsulfonyl)phenyl]-2-[(2-pyridinylmethyl)amino]-3-pyridinecarbonitrile;
4-ethyl-2-{[(5-methyl-2-pyridinyl)methyl]amino}-6-[4-(methylsulfonyl)phenyl]-3-pyridinecarbonitrile;
4-ethyl-2-{[(6-methyl-3-pyridinyl)methyl]amino}-6-[4-(methylsulfonyl)phenyl]-3-pyridinecarbonitrile;
4-ethyl-2-{[(1-methyl-1H-pyrazol-4-yl)methyl]amino}-6-[4-(methylsulfonyl)phenyl]-3-pyridinecarbonitrile;
4-ethyl-6-[4-(methylsulfonyl)phenyl]-2-{[(4-methyl-1,3-thiazol-2-yl)methyl]amino}-3-pyridinecarbonitrile;
4-ethyl-6-[4-(methylsulfonyl)phenyl]-2-[(2-pyridinylmethyl)oxy]-3-pyridinecarbonitrile;
4-ethyl-N-[(1-ethyl-1H-1,2,4-triazol-5-yl)methyl]-6-[4-(methylsulfonyl)phenyl]-2-pyridinamine;
4-ethyl-2-{[(6-methyl-3-pyridinyl)methyl]oxy}-6-[4-(methylsulfonyl)phenyl]-3-pyridinecarbonitrile;
6-[4-(methylsulfonyl)phenyl]-N-[(1-methyl-1H-1,2,4-triazol-5-yl)methyl]-4-(trifluoromethyl)-2-pyridinamine; and pharmaceutically acceptable salts and solvates thereof.

6. The method according to claim 5, wherein the compound is 2-butoxy-4-[4-(methylsulfonyl)phenyl]-6-(trifluoromethyl)pyrimidine or a pharmaceutical acceptable salt or solvate thereof.

7. The method according to claim 4, characterised in that the selective serotonin reuptake inhibitor is selected from citalopram, escitalopram, fluoxetine, R-fluoxetine, sertraline, paroxetine, fluvoxamine, venlafaxine, duloxetine, dapoxetine, nefazodone, imipramine, imipramine N-oxide, desipramine, pirandamine, dazepinil, nefopam, befuraline, fezolamine, femoxetine, clomipramine, cianoimipramine, litoxetine, cericlamine, seproxetine, WY 27587, WY 27866, imeldine, ifoxetine, tiflucarbine, viqualine, milnacipran, bazinaprine, YM 922, S 33005, F 98214-TA, OPC 14523, alaproclate, cyanodothepine, trimipramine, quinupramine, dothiepin, amoxapine, nitroxazepine, McN 5652, McN 5707, VN 2222, L 792339, roxindole, YM 35992,0177, Org 6582, Org 6997, Org 6906, amitriptyline, amitriptyline N-oxide, nortriptyline, CL 255.663, pirlindole, indatraline, LY 113.821, LY 214.281, CGP 6085 A, RU 25.591, napamezole, diclofensine, trazodone, EMD 68.843, BMY 42.569, NS 2389, sercloremine, nitroquipazine, ademethionine, sibutramine, clovoxamine, and mixtures thereof.

8. The method according to claim 7, wherein the selective serotonin reuptake inhibitor is paroxetine.

9. The method of claim 5, wherein the compound is 2-butoxy-4-[4-(methylsulfonyl)phenyl]-6-(trifluoromethyl)pyrimidine or a pharmaceutical acceptable salt thereof, further comprising combination with paroxetine.

10. The method of claim 2, further comprising combination with selective serotonin reuptake inhibitor.

11. The method according to claim 10, wherein said mammal is human.

12. The method according to claim 11, wherein said depressive disorder is selected from the group: bipolar disorder, bipolar depression, bipolar disorder I, bipolar disorder II, unipolar depression.

13. The method according to claim 10, wherein said selective serotonin reuptake inhibitor is selected from citalopram, escitalopram, fluoxetine, R-fluoxetine, sertraline, paroxetine, fluvoxamine, venlafaxine, duloxetine, dapoxetine, nefazodone, imipramine, imipramine N-oxide, desipramine, pirandamine, dazepinil, nefopam, befuraline, fezolamine, femoxetine, clomipramine, cianoimipramine, litoxetine, cericlamine, seproxetine, WY 27587, WY 27866, imeldine, ifoxetine, tiflucarbine, viqualine, milnacipran, bazinaprine, YM 922, S 33005, F 98214-TA, OPC 14523, alaproclate, cyanodothepine, trimipramine, quinupramine, dothiepin, amoxapine, nitroxazepine, McN 5652, McN 5707, VN 2222, L 792339, roxindole, YM 35992,0177, Org 6582, Org 6997, Org 6906, amitriptyline, amitriptyline N-oxide, nortriptyline, CL 255.663, pirlindole, indatraline, LY 113.821, LY 214.281, CGP 6085 A, RU 25.591, napamezole, diclofensine, trazodone, EMD 68.843, BMY 42.569, NS 2389, sercloremine, nitroquipazine, ademethionine, sibutramine, clovoxamine, and mixtures thereof.

14. The method according to claim 13, wherein said selective serotonin reuptake inhibitor is paroxetine.

15-16. (canceled)

17. The method according to claim 4, wherein said mammal is human.

18. The method according to claim 17, wherein said depressive disorder is selected from the group: bipolar disorder, bipolar depression, bipolar disorder I, bipolar disorder II, unipolar depression.

19. The method of claim 3, further comprising combination with a selective serotonin reuptake inhibitor.

20. The method according to claim 19, wherein said mammal is human.

21. The method according to claim 19, wherein said depressive disorder is selected from the group: bipolar disorder, bipolar depression, bipolar disorder I, bipolar disorder II, unipolar depression.

22. The method according to claim 19, wherein said selective serotonin reuptake inhibitor is selected from citalopram, escitalopram, fluoxetine, R-fluoxetine, sertraline, paroxetine, fluvoxamine, venlafaxine, duloxetine, dapoxetine, nefazodone, imipramine, imipramine N-oxide, desipramine, pirandamine, dazepinil, nefopam, befuraline, fezolamine, femoxetine, clomipramine, cianoimipramine, litoxetine, cericlamine, seproxetine, WY 27587, WY 27866, imeldine, ifoxetine, tiflucarbine, viqualine, milnacipran, bazinaprine, YM 922, S 33005, F 98214-TA, OPC 14523, alaproclate, cyanodothepine, trimipramine, quinupramine, dothiepin, amoxapine, nitroxazepine, McN 5652, McN 5707, VN 2222, L 792339, roxindole, YM 35992,0177, Org 6582, Org 6997, Org 6906, amitriptyline, amitriptyline N-oxide, nortriptyline, CL 255.663, pirlindole, indatraline, LY 113.821, LY 214.281, CGP 6085 A, RU 25.591, napamezole, diclofensine, trazodone, EMD 68.843, BMY 42.569, NS 2389, sercloremine, nitroquipazine, ademethionine, sibutramine, clovoxamine, and mixtures thereof.

23. The method according to claim 22, wherein said selective serotonin reuptake inhibitor is paroxetine.
